(19) United States
(12) Patent Application Publication
(10) Pub. No.: US 2019/0354814 A1
(43) Pub. Date: Nov. 21, 2019

(54) SYSTEMS AND METHODS FOR USING SUPERVISED LEARNING TO PREDICT SUBJECT-SPECIFIC BACTEREMIA OUTCOMES

(71) Applicant: Henry M. Jackson Foundation for the Advancement of Military Medicine, Bethesda, MD (US)

(72) Inventors: Seth A. Schobel, Clarksburg, MD (US); Eric A. Elster, Kensington, MD (US); Beverly J. Gaucher, Bethesda, MD (US)

(73) Assignee: Henry M. Jackson Foundation for the Advancement of Military Medicine, Bethesda, MD (US)

(21) Appl. No.: 16/476,144

(22) PCT Filed: Jan. 5, 2018
(86) PCT No.: PCT/US2018/012708

§ 371 Date: Jul. 5, 2019

Related U.S. Application Data
(60) Provisional application No. 62/445,690, filed on Jan. 12, 2017.
(60) Provisional application No. 62/443,780, filed on Jan. 8, 2017.

(51) Int. Cl. G06K 9/62; G16H 50/20; G06N 20/20; G06N 3/08

(52) U.S. Cl. CPC G06K 9/6269; G16H 50/20; G06K 9/6278; G06N 3/08; G06K 9/6276; G06N 20/20

(57) ABSTRACT

Described herein are systems and methods for determining if a subject has an increased risk of having or developing bacteremia or symptoms associated with bacteremia. Also described are systems and methods for predicting a bacteremia outcome for a subject, systems and methods for generating a model for predicting a bacteremia outcome in a subject, systems and method for determining a subject's risk profile for bacteremia, method of determining that a subject has an increased risk of developing bacteremia, and methods of treating a subject determined to have an elevated risk of developing bacteremia, methods of detecting panels of biomarkers in a subject, and methods of assessing risk factors in a subject having an injury, as well as related devices and kits.

### CROSS-REFERENCE TO RELATED APPLICATIONS

This application claims the benefit of and priority to U.S. Provisional Application No. 62/443,780, filed Jan. 8, 2017, titled “PREDICTIVE BIOMARKERS FOR BACTEREMIA AND/OR PNEUMONIA” and U.S. Provisional Application No. 62/445,690, filed Jan. 12, 2017, titled “PREDICTIVE FACTORS FOR BACTEREMIA AND/OR PNEUMONIA”, the entire disclosures of which are incorporated herein in their entireties for any and all purposes.

### STATEMENT REGARDING FEDERALLY SPONSORED RESEARCH OR DEVELOPMENT

This invention was made with government support under HT9404-13-1-0032 and HU0001-15-2-0001 awarded by the Uniformed Services University. The government has certain rights in the invention.

### FIELD

Described herein are systems and methods for determining if a subject has an increased risk of having or developing bacteremia or symptoms associated with bacteremia. Also described are systems and methods for predicting a bacteremia outcome for a subject, systems and methods for generating a model for predicting a bacteremia outcome in a subject, systems and method for determining a subject's risk profile for bacteremia, method of determining that a subject has an increased risk of developing bacteremia, and methods of treating a subject determined to have an elevated risk of developing bacteremia, methods of detecting panels of biomarkers in a subject, and methods of assessing risk factors in a subject having an injury, as well as related devices and kits.

### BACKGROUND

Nosocomial infections are common occurrences in critically ill patients. Indeed, patients requiring intensive care unit (ICU) level of care have a three to five fold increase in these morbid complications. These infections remain the leading cause of late death after traumatic injury. One of the most common complications that inflict critically ill and injured patients is bacteremia. Up to 10% of ICU patients experience a blood stream infection, many of which are related to indwelling catheters.

While much of the focus on the late care of the ICU patient involves diagnosis and management of infections, less work has been done around prediction and risk stratification. While preventative strategies and guidelines are now widely published, much of the care of the patient who develops a nosocomial infection remains reactive. Having tools that would allow a bedside clinician to predict or identify the patients at highest risk for a variety of infectious complications could allow for more proactive and directed preventative strategies. Indeed, recent emphasis on precision medicine and a recent Institute of Medicine Report on the current rate of diagnostic error suggest that there is a great need to improve the timeliness and accuracy of predictive and diagnostic methods in ICU patients.

### SUMMARY

Described herein are methods of determining if a subject has an increased risk of having or developing bacteremia or symptoms associated with bacteremia, including prior to the detection of symptoms thereof and/or prior to onset of any detectable symptoms thereof, methods for predicting bacteremia outcomes, and related methods of treatment. Advantages of such early detection and/or treatment may include avoidance of sepsis, avoidance of empyema, avoidance of need for ventilation support, reduced length of stay in hospital or intensive care unit, and/or reduced medical costs.

The present disclosure also provides methods of treating individuals determined to have an increased risk of developing bacteremia, optionally before the onset of detectable symptoms thereof, such as before there are perceivable, noticeable or measurable signs of bacteremia in the individual. Examples of treatment may include initiation or broadening of antibiotic therapy. Benefits of such early treatment may include avoidance of sepsis, empyema, need for ventilation support, reduced length of stay in hospital or intensive care unit, and/or reduced medical costs.

In accordance with some embodiments, there are provided methods for predicting a bacteremia outcome for a subject. The methods include receiving, by one or more processors, for each of a plurality of first subjects, a first value of at least one clinical parameter of a plurality of clinical parameters and a corresponding bacteremia outcome; generating, by the one or more processors, a training database associating the first values of the plurality of clinical parameters to the corresponding bacteremia outcomes of the plurality of first subjects; executing, by the one or more processors, a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes; executing, by the one or more processors, for each subset of model parameters, a classification algorithm to generate predictions of bacteremia outcomes based on the subset of model parameters; calculating, by the one or more processors, for each classification algorithm executed based on each corresponding subset of model parameters, at least one performance metric indicative of a level of performance of the classification algorithm and the corresponding subset of model parameters in predicting bacteremia outcomes; selecting, by the one or more processors, a candidate classification algorithm and corresponding subset of model parameters based on the at least one performance metric of the candidate classification algorithm and corresponding subset of model parameters; receiving, by the one or more processors, for at least one second subject, a second value of the at least one clinical parameter of the plurality of clinical parameters; executing, by the one or more processors, the selected candidate classification algorithm using the corresponding subset of model parameters and the second value of the at least one clinical parameter to calculate a predicted outcome for bacteremia specific to the at least one second subject; and outputting, by the one or more processors, the predicted outcome for bacteremia specific to the at least one second subject.

In accordance with some embodiments, there are provided methods for generating a model for predicting a bacteremia outcome for a subject. The methods include receiving, by one or more processors, for each of a plurality of first subjects, a first value of at least one clinical parameter of a plurality of clinical parameters and a corresponding bacteremia outcome; generating, by the one or more processors, a training database associating the first values of the plurality of clinical parameters to the corresponding bacteremia outcomes of the plurality of first subjects; executing, by the one or more processors, a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes; executing, by the one or more processors, for each subset of model parameters, a classification algorithm to generate predictions of bacteremia outcomes based on the subset of model parameters; calculating, by the one or more processors, for each classification algorithm executed based on each corresponding subset of model parameters, at least one performance metric indicative of a level of performance of the classification algorithm and the corresponding subset of model parameters in predicting bacteremia outcomes; selecting, by the one or more processors, a candidate classification algorithm and corresponding subset of model parameters based on the at least one performance metric of the candidate classification algorithm and corresponding subset of model parameters; and outputting, by the one or more processors, the candidate classification algorithm and corresponding subset of model parameters.

In accordance with some embodiments, there are provided methods for predicting a bacteremia outcome for a subject. The methods include receiving, for a second subject, a second value of at least one clinical parameter of a plurality of clinical parameters; executing a classification algorithm using the second value of the at least one clinical parameter of the first subject to predict a bacteremia outcome specific to the first subject, the classification algorithm selected by using a plurality of variable selection algorithms to select subsets of model parameters from the plurality of clinical parameters, the subsets of model parameters representing nodes of Bayesian networks indicating conditional dependencies between the subsets of model parameters and corresponding bacteremia outcomes, the variable selection algorithms executed using first values of the plurality of clinical parameters for a plurality of first subjects and corresponding bacteremia outcomes, the classification algorithm selected further based on performance metrics indicative of an ability of the classification algorithm to predict bacteremia outcomes; and outputting the predicted bacteremia outcome specific to the second subject.

In specific embodiments of any of these methods, the subjects have an injury that puts the subject at risk of developing bacteremia, such as a blast injury, a crush injury, a gunshot wound, or an extremity wound.

In specific embodiments of any of these methods, the predicted bacteremia outcomes generated by the candidate classification algorithm using the corresponding subset of model parameters includes at least one of (i) an indication that the second subject has bacteremia or (ii) an indication that the second subject is at risk for developing bacteremia; and the bacteremia outcome received for each first subject is based on a confirmed presence of bacteria in the blood diagnosed through isolation of a pathogen from at least one quantitated blood culture.

In specific embodiments of any of these methods, each first subject has an injury that puts the subject at risk of developing bacteremia, and the clinical parameters for which values are received for each first subject include at least one selected from gender, age, date of injury, location of injury, presence of abdominal injury, mechanism of injury, wound depth, wound surface area, number of wound debridements, associated injuries, type of wound closure, success of wound closure, requirement for transfusion, total number of blood products transfused, amount of whole blood cells administered to the subject, amount of red blood cells (RBCs) administered to the subject, amount of packed red blood cells (pRBCs) administered to the subject, amount of platelets administered to the subject, level of total packed RBCs, Injury Severity Score (ISS), AIS of abdomen, AIS of head, AIS of chest (thorax), Acute Physiology and Chronic Health Evaluation II (APACHE II) score, presence of critical colonization (CC) in a sample from the subject, presence of traumatic brain injury, severity of traumatic brain injury, length of hospital stay, length of intensive care unit (ICU) stay, number of days on a ventilator, disposition from hospital, development of nosocomial infections, level of interferon gamma induced protein 10 (IP-10) in a sample from the subject, level of soluble interleukin 2 receptor (IL-2R) in a sample from the subject, level of interleukin-10 (IL-10) in a sample from the subject, level of interleukin-3 (IL-3) in a sample from the subject, level of interleukin-6 (IL-6) in a sample from the subject, level of interleukin-7 (IL-7) in a sample from the subject, level of interleukin-8 (IL-8) in a sample from the subject, level of monocyte chemoattractant protein 1 (MCP-1) in a sample from the subject, level of monokine induced by gamma interferon (MIG) in a sample from the subject, and level of eotaxin in a sample from the subject.

In specific embodiments of any of these methods, the clinical parameters for which values are received for each first subject include at least one selected from a biomarker clinical parameter, an administration of blood products clinical parameter, or an injury severity score clinical parameter.

In specific embodiments of any of these methods, the clinical parameters include at least one level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-1α) in a sample from the subject, level of interleukin 1 beta (IL-1β) in a sample from the subject, level of interleukin 1 receptor antagonist (IL-IRA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (pRBCs) administered to the subject, amount of platelets administered to the subject, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, or AIS of skin.

In specific embodiments of any of these methods, the clinical parameters for which values are received for each first subject include at least one selected from Luminex proteomic data, RNAseq, transcriptomic data, quantitative polymerase chain reaction (qPCR) data, and quantitative bacteriology data.

In specific embodiments of any of these methods, the clinical parameters for which values are received for each second subject include at least one selected from a presence of CC in a sample from the subject, level of total RBCs administered to the subject, summation of all blood products administered to the subject, level of IL-2R in a serum sample from the subject, and level of MIG in a serum sample from the subject.

In specific embodiments of any of these methods, the subset of model parameters corresponding to the candidate classification algorithm include at least two selected from a presence of CC in a sample from the subject, level of total RBCs administered to the subject, summation of all blood products administered to the subject, level of IL-2R in a serum sample from the subject, and level of MIG in a serum sample from the subject.

In specific embodiments of any of these methods, the at least one performance metric includes at least one of a total out-of-bag (OOB) error estimate, a positive class OOB error estimate, a negative class OOB error estimate, an accuracy score, or a Kappa score.

In specific embodiments of any of these methods, selecting the candidate classification algorithm and corresponding subset of model parameters includes executing a decision curve analysis (DCA) with each classification algorithm, the DCA indicating a net benefit of providing a treatment based on bacteremia outcomes generated by the classification algorithm, and selecting the classification algorithm having a largest net benefit of providing the treatment.

In specific embodiments of any of these methods, the methods can include using the DCA to compare the predicted bacteremia outcome for the at least one second subject to a specified risk threshold to determine the net benefit of treatment.

In specific embodiments of any of these methods, candidate classification algorithm is a naïve Bayes model.

In specific embodiments of any of these methods, for each first subject, first values are received for at least two clinical parameters, the first values corresponding to a single point in time.

In specific embodiments of any of these methods, the methods can include identifying at least one first subject for which a count of clinical parameters for which values are received is less than the count of the training parameters; and executing an imputation algorithm to generate an imputed value for at least one of the training parameters corresponding to a clinical parameter associated with the at least one first subject for which a value is not received.

In specific embodiments of any of these methods, the plurality of variable selection algorithms include at least two of an inter.iamb algorithm, a fast.iamb algorithm, an iamb algorithm, a gs algorithm, an mmpc algorithm, or a si.hiton.pc algorithm.

In accordance with some embodiments, there are provided systems for predicting a bacteremia outcome in a subject. The systems include a processing circuit including one or more processor and a memory, and a display device. The memory includes a training database, a machine learning engine, and a prediction engine. The training database is configured to store, for each of a plurality of first subjects, a first value of at least one clinical parameter of a plurality of clinical parameters and a corresponding bacteremia outcome. The machine learning engine is configured to execute a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes. The machine learning engine is configured to execute, for each subset of model parameters, a classification algorithm to generate predictions of bacteremia outcomes based on the subset of model parameters. The machine learning engine is configured to select a candidate classification algorithm and corresponding subset of model parameters based on the at least one performance metric of the candidate classification algorithm and the corresponding subset of model parameters. The prediction engine is configured to receive, for at least one second subject, a second value of at least one clinical parameter of the plurality of clinical parameters. The machine learning executes the selected candidate classification algorithm using the corresponding subset of model parameters and the second value of the at least one clinical parameter to calculate a predicted outcome for bacteremia specific to the at least one second subject. The display device displays the predicted outcome for bacteremia specific to the at least one second subject.

In accordance with some embodiments, there are provided systems for generating a model for predicting a bacteremia outcome in a subject. The systems include a processing circuit including one or more processors and a memory. The memory includes a training database and a machine learning engine. The training database is configured to store, for each of a plurality of first subjects, a first value of at least one clinical parameter of a plurality of clinical parameters and a corresponding bacteremia outcome. The machine learning engine is configured to execute a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes; execute, for each subset of model parameters, a classification algorithm to generate predictions of bacteremia outcomes based on the subset of model parameters; calculate, for each classification algorithm executed based on each corresponding subset of model parameters, at least one performance metric indicative a level of performance of the classification algorithm and the corresponding subset of model parameters in predicting bacteremia outcomes; select a candidate classification algorithm and corresponding subset of model parameters based on the at least one performance metric of the candidate classification algorithm and the corresponding subset of model parameters; and output the candidate classification algorithm and corresponding subset of model parameters.

In accordance with some embodiments, there are provided systems for predicting a bacteremia outcome in a subject. The systems include a processing circuit including one or more processor and a memory, and a display device. The memory includes a prediction engine configured to receive, for a second subject, a second value of at least one clinical parameter of a plurality of clinical parameters; and execute a classification algorithm using the second value of the at least one clinical parameter of the second subject to predict a bacteremia outcome specific to the second subject, the classification algorithm selected by using a plurality of variable selection algorithms to select subsets of model parameters from the plurality of clinical parameters, the subsets of model parameters representing nodes of Bayesian networks indicating conditional dependencies between the subsets of model parameters and corresponding bacteremia outcomes, the variable selection algorithms executed using first values of the plurality of clinical parameters for a plurality of first subjects and corresponding bacteremia outcomes, the classification algorithm selected further based on performance metrics indicative of an ability of the classification algorithm to predict bacteremia outcomes. The display device is configured to output the predicted bacteremia outcome specific to the second subject.

In accordance with some embodiments, there are provided non-transient computer-readable media including computer-executable instructions stored thereon, which when executed by one or more processors, cause the one or more processors to receive, for each of a plurality of first subjects, a first value of at least one clinical parameter of a plurality of clinical parameters and a corresponding bacteremia outcome; generate a training database associating the first values of the plurality of clinical parameters to the corresponding bacteremia outcomes of the plurality of first subjects; execute a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes; execute, for each subset of model parameters, a classification algorithm to generate predictions of bacteremia outcomes based on the subset of model parameters; calculate, for each classification algorithm executed based on each corresponding subset of model parameters, at least one performance metric indicative of a level of performance of the classification algorithm and the corresponding subset of model parameters in predicting bacteremia outcomes; select a candidate classification algorithm and corresponding subset of model parameters based on the at least one performance metric of the candidate classification algorithm and corresponding subset of model parameters; receive, for at least one second subject, a second value of the at least one clinical parameter of the plurality of clinical parameters, execute the selected candidate classification algorithm using the corresponding subset of model parameters and the second value of the at least one clinical parameter to calculate a predicted outcome for bacteremia specific to the at least one second subject; and output the predicted outcome for bacteremia specific to the at least one second subject.

In accordance with some embodiments, there are provided non-transient computer-readable media including computer-executable instructions stored thereon, which when executed by one or more processors, cause the one or more processors to store, for each of a plurality of first subjects, a first value of at least one clinical parameter of a plurality of clinical parameters and a corresponding bacteremia outcome; execute a plurality of variable selection algorithms to select a subset of model parameters from the plurality of clinical parameters for each variable selection algorithm, wherein a count of each subset of model parameters is less than a count of the plurality of clinical parameters, and each subset of model parameters represent nodes of a Bayesian network indicating conditional dependencies between the subset of model parameters and the corresponding bacteremia outcomes; execute, for each subset of model parameters, a classification algorithm to generate predictions of bacteremia outcomes based on the subset of model parameters; calculate, for each classification algorithm executed based on each corresponding subset of model parameters, at least one performance metric indicative a level of performance of the classification algorithm and the corresponding subset of model parameters in predicting bacteremia outcomes; select a candidate classification algorithm and corresponding subset of model parameters based on the at least one performance metric of the candidate classification algorithm and the corresponding subset of model parameters; and output the candidate classification algorithm and corresponding subset of model parameters.

In accordance with some embodiments, there are provided non-transient computer-readable media including computer-executable instructions stored thereon, which when executed by one or more processors, cause the one or more processors to receive, for a second subject, a second value of at least one clinical parameter of a plurality of clinical parameters; execute a classification algorithm using the second value of the at least one clinical parameter of the second subject to predict a bacteremia outcome specific to the second subject, the classification algorithm selected by using a plurality of variable selection algorithms to select subsets of model parameters from the plurality of clinical parameters, the subsets of model parameters representing nodes of Bayesian networks indicating conditional dependencies between the subsets of model parameters and corresponding bacteremia outcomes, the variable selection algorithms executed using first values of the plurality of clinical parameters for a plurality of first subjects and corresponding bacteremia outcomes, the classification algorithm selected further based on performance metrics indicative of an ability of the classification algorithm to predict bacteremia outcomes; and cause a display device to output the predicted bacteremia outcome specific to the second subject.

In accordance with some embodiments, there are provided methods of determining a risk profile for bacteremia, optionally prior to the onset of detectable symptoms thereof, in a subject having an injury that puts the subject at risk of developing bacteremia, wherein the risk profile comprises one or more components based on one or more clinical parameters selected a presence of CC in a sample from the subject, level of total RBCs administered to the subject, summation of all blood products administered to the subject, level of IL-2R in a serum sample from the subject, and level of MIG in a serum sample from the subject. The methods include detecting the one or more clinical parameters for the subject, and calculating a value of the risk profile of the subject from the detected clinical parameters.

In accordance with some embodiments, there are provided methods of determining that a subject having an injury that puts the subject at risk of developing bacteremia has an increased risk of developing bacteremia, optionally prior to the onset of detectable symptoms thereof. The methods include detecting one or more clinical parameters for the subject selected from a presence of CC in a sample from the subject, level of total RBCs administered to the subject, summation of all blood products administered to the subject, level of IL-2R in a serum sample from the subject, and level of MIG in a serum sample from the subject, calculating a value of the risk profile of the subject from the detected clinical parameters; and comparing the value of the risk profile of the subject to a reference risk profile value, wherein an increase in the value of the risk profile of the subject as compared to the reference risk profile value indicates that the subject has an increased risk of developing bacteremia.

In accordance with some embodiments, there are provided methods of treating a subject having an injury that puts the subject at risk of developing bacteremia for bacteremia. The methods include administering a treatment for bacteremia to the subject prior to the onset of detectable symptoms thereof, wherein the subject previously has been determined to have an elevated risk of developing bacteremia as determined by a risk profile value calculated from one or more clinical parameters selected from a presence of CC in a sample from the subject, level of total RBCs administered to the subject, summation of all blood products administered to the subject, level of IL-2R in a serum sample from the subject, and level of MIG in a serum sample from the subject.

In specific embodiments of any of these methods, an increase in the subject's risk profile value as compared to the reference risk profile value indicates that the subject has an increased risk of developing bacteremia.

In specific embodiments of any of these methods, the reference risk profile value is calculated from clinical parameters previously detected for the subject.

In specific embodiments of any of these methods, the reference risk profile value is calculated from clinical parameters previously detected for the subject at a time the subject has the injury.

In specific embodiments of any of these methods, the reference risk profile value is calculated from clinical parameters detected for a population of reference subjects having an injury at a time when the reference subjects did not have detectable symptoms of bacteremia.

In specific embodiments of any of these methods, the methods are conducted prior to the onset of detectable symptoms of bacteremia in the subject.

In specific embodiments of any of these methods, one or more clinical parameters are detected in a sample from the subject selected from a serum sample and wound effluent.

In accordance with some embodiments, there are provided methods of detecting levels of biomarkers in a subject having an injury. The methods include measuring in one or more samples from the subject levels of one or more biomarkers selected from IL-2R and MIG. The methods can include measuring levels of IL-2R and MIG.

In accordance with some embodiments, there are provided methods of assessing risk factors in a subject having an injury that puts the subject at risk of developing bacteremia. The methods include assessing one or more risk factors selected from a presence of CC in a sample from the subject, level of total RBCs administered to the subject, summation of all blood products administered to the subject, level of IL-2R in a serum sample from the subject, and level of MIG in a serum sample from the subject in a serum sample from the subject.

In accordance with some embodiments, there are provided kits for performing any of these methods.

In accordance with some embodiments, there are provide antibiotics, for treating bacteremia in a subject having an injury that puts the subject at risk of developing bacteremia, prior to the onset of detectable symptoms thereof, wherein the subject previously has been determined to have an elevated risk of developing bacteremia as determined according to any of these methods.

In accordance with some embodiments, there are provided uses of an antibiotic in the preparation of a medicament for treating bacteremia in a subject having an injury that puts the subject at risk of developing bacteremia, prior to the onset of detectable symptoms thereof, wherein the subject previously has been determined to have an elevated risk of developing bacteremia as determined by any of these methods.

### BRIEF DESCRIPTION OF THE DRAWINGS

FIG. 1 illustrates a block diagram of an embodiment of a clinical outcome prediction system (“COPS”) for predicting subject-specific bacteremia outcomes as described herein.

FIG. 2 illustrates an embodiment of a Bayesian Network as described herein and the model parameters representing the nodes of the Bayesian Network to indicate conditionally dependent relationships between the model parameters and the bacteremia outcomes, the model parameters selected using the COPS of FIG. 1.

FIG. 3 illustrates an embodiment of a decision tree from a random forest model generated using the model parameters selected using the COPS of FIG. 1.

FIG. 4 illustrates an embodiment of a chart of performance metrics of a candidate classification algorithm and corresponding model parameters of a Bayesian network as selected by the COPS of FIG. 1.

FIG. 5 illustrates an embodiment of a Decision Curve Analysis for a candidate classification algorithm and corresponding model parameters of a Bayesian network as selected by the COPS of FIG. 1.

FIG. 6 illustrates an embodiment of method for predicting subject-specific bacteremia outcomes as described herein.

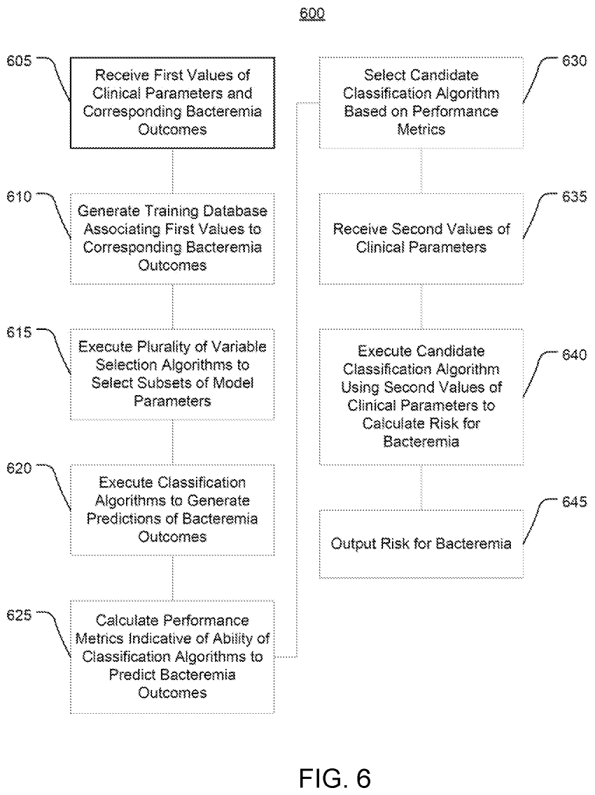

### DETAILED DESCRIPTION

### Definitions

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject and is not limited to any specific dosage forms or routes of administration unless otherwise stated.

The terms “treat”, “treating” or “treatment”, as used herein, include alleviating, abating or ameliorating bacteremia or one or more symptoms thereof, whether or not bacteremia is considered to be “cured” or “healed” and whether or not all symptoms are resolved. The terms also include reducing or preventing progression of bacteremia or one or more symptoms thereof, impeding or preventing an underlying mechanism of bacteremia or one or more symptoms thereof, and achieving any therapeutic and/or prophylactic benefit.

As used herein, the term “subject,” “patient,” or “test subject” indicates a mammal, in particular a human or non-human primate. The test subject may or may not be in need of an assessment of a predisposition to bacteremia. In some embodiments, the test subject is assessed prior to the detection of symptoms of bacteremia, such as prior to detection of the presence of bacteria in the subject's blood system, such as by one or more of White Blood Cell (WBC) count, Absolute Neutrophil Count (ANC), Absolute Band Count (ABC), Erythrocyte Sedimentation Rate (ESR), C-Reactive Protein (CRP) level, Procalcitonin level, Urinalysis and Urine culture, Stool studies for children with diarrhea, Plasma Clearance Rate, Lumbar puncture and cerebrospinal fluid (CSF) analysis and Blood culture. In some embodiments, the test subject is assessed prior to the onset of any detectable symptoms of bacteremia, such as prior to the subject detectable levels of bacteria in the subject's blood system, such as may be detectable by one or more of White Blood Cell (WBC) count, Absolute Neutrophil Count (ANC), Absolute Band Count (ABC), Erythrocyte Sedimentation Rate (ESR), C-Reactive Protein (CRP) level, Procalcitonin level, Urinalysis and Urine culture, Stool studies for children with diarrhea, Plasma Clearance Rate, Lumbar puncture and cerebrospinal fluid (CSF) analysis and Blood culture. In some embodiments, the test subject does not have detectable symptoms of any type of sickness or condition. In some embodiments, the test subject has an injury, condition, or wound that puts the subject at risk of developing bacteremia, such as having a viral or bacterial infection, such as but not limited to urinary tract infection, meningitis, pericarditis, endocarditis, osteomyelitis, and infectious arthritis, having or developing bronchitis, undergoing a medical surgical or dental procedure, having an open wound or trauma, such as but not limited to a wound received in combat, a blast injury, a crush injury, a gunshot wound, an extremity wound, suffering a nosocomial infection, having undergone medical interventions such as central line placement or intubation, having diabetes, having HIV, undergoing hemodialysis, undergoing organ transplant procedure (donor or receiver), receiving a glucocorticoid or any other immunosuppressive treatments, such as but not limited to calcineurin inhibitors, mTOR inhibitors, IMDH inhibitors and biologics or monoclonal antibodies. In some embodiments, the subject does not have a condition that puts the subject at risk of developing bacteremia, prior to application of the methods described herein. In other embodiments, the subject has a condition that puts the subject at risk of developing bacteremia.

The term “bacteremia” is used herein as it is in the art and means the presence of bacteria in the subject's blood system. Bacteremia may or may not have any discernable symptoms prior to a successful diagnosis thereof. Symptoms of bacteremia include but are not limited to fever, rapid heart rate, shaking chills, low blood pressure, gastrointestinal symptoms, such as but not limited to abdominal pain, nausea, vomiting, and diarrhea, rapid breathing, and/or confusion. If severe enough, bacteremia can lead to sepsis, severe sepsis and possible septic shock. Accordingly, the term bacteremia, as used herein, includes non-septic bacterial infection of the blood as well as septic bacterial blood infections. In some embodiments, the bacteremia that is treated or tested for is not sepsis, severe sepsis or septic shock. In other embodiments, the bacteremia that is treated or tested for is sepsis, severe sepsis or septic shock.

Bacteremia may, but need not, be diagnosed at any point during the application of the methods of the present disclosure. In one embodiment, bacteremia diagnostic tests are performed on the subject after the application of the methods of the present disclosure. Current methods of diagnosing, but not predicting the onset of, bacteremia include but are not limited to, White Blood Cell (WBC) count, Absolute Neutrophil Count (ANC), Absolute Band Count (ABC), Erythrocyte Sedimentation Rate (ESR), C-Reactive Protein (CRP) level, Procalcitonin level, Urinalysis and Urine culture, Stool studies for children with diarrhea, Plasma Clearance Rate, Lumbar puncture and cerebrospinal fluid (CSF) analysis, and Blood culture. Any one of these diagnostic procedures can be performed prior to applying the methods of the present disclosure to the subject to confirm that the subject does not presently have bacteremia. Additionally or alternatively, such bacteremia diagnostic procedures may be performed after applying the methods of the present disclosure to the subject. Such “post method” bacteremia diagnostic procedures may be useful in monitoring the early onset of bacteremia before the development of any discernible symptoms.

As used herein, the term “increased risk” or “elevated risk” is used to mean that the test subject has an increased chance of developing or acquiring bacteremia compared to a normal or reference individual or population of individuals. In some embodiments, the reference individual is the test subject at an earlier time point, including prior to having an injury, condition, or wound that puts the subject at risk of developing bacteremia, or at an earlier point in time after having such an injury, condition, or wound. The increased risk may be relative or absolute and may be expressed qualitatively or quantitatively. For example, an increased risk may be expressed as simply determining the subject's risk profile and placing the subject in an “increased risk” category, based upon previous studies. Alternatively, a numerical expression of the subject's increased risk may be determined based upon the risk profile. As used herein, examples of expressions of an increased risk include but are not limited to, odds, probability, odds ratio, p-values, attributable risk, biomarker index score, relative frequency, positive predictive value, negative predictive value, and relative risk. Risk may be determined based on predicting bacteremia outcomes for the subject; for example, a predicted bacteremia outcome may include an indication of whether the subject has bacteremia or does not have bacteremia, an indication of a likelihood that the subject has bacteremia or does not have bacteremia, or an indication of a likelihood that the subject will contract bacteremia.

For example, the correlation between a subject's risk profile and the likelihood of suffering from bacteremia may be measured by an odds ratio (OR) and by the relative risk (RR). If P(R⁺) is the probability of developing bacteremia for individuals with the risk profile (R) and P(R⁻) is the probability of developing bacteremia for individuals without the risk profile, then the relative risk is the ratio of the two probabilities: RR=P(R⁺)/P(R⁻).

In case-control studies, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a risk profile in cases studies and F is the frequency of risk profile in controls. F⁺ and F can be calculated using the risk profile frequencies of the study.

The attributable risk (AR) can also be used to express an increased risk. The AR describes the proportion of individuals in a population exhibiting bacteremia to a specific member of the risk profile. AR may also be important in quantifying the role of individual components (specific member) in condition etiology and in terms of the public health impact of the individual risk factor. The public health relevance of the AR measurement lies in estimating the proportion of cases of bacteremia in the population that could be prevented if the profile or individual factor were absent. AR may be determined as follows: AR=P_{E}(RR−1)/(P_{E}(RR−1)+1), where AR is the risk attributable to a profile or individual factor of the profile, and P_{E} is the frequency of exposure to a profile or individual component of the profile within the population at large. RR is the relative risk, which can be approximated with the odds ratio when the profile or individual factor of the profile under study has a relatively low incidence in the general population.

Associations with specific profiles can be performed using regression analysis by regressing the risk profile with the presence or absence of diagnosed bacteremia. The regression may or may not be corrected or adjusted for one or more factors. The factors for which the analyses may be adjusted include, but are not limited to age, sex, weight, ethnicity, type of wound if present, geographic location, fasting state, state of pregnancy or post-pregnancy, menstrual cycle, general health of the subject, alcohol or drug consumption, caffeine or nicotine intake, and circadian rhythms.

### A. Factors, Biomarkers, Clinical Parameters, and Components

The terms “factor,” “risk factor,” and/or “component” are used herein to refer to individual constituents that are assessed, detected, measured, received, and/or determined prior to or during the performance of any of the methods described herein. For convenience, they are referred to herein as clinical parameters.

Examples of clinical parameters of a subject include, but are not limited to any one or more of gender, age, date of injury, location of injury, presence of abdominal injury, mechanism of injury, wound depth, wound surface area, number of wound debridements, associated injuries, type of wound closure, success of wound closure, requirement for transfusion, total number of blood products transfused, amount of whole blood cells administered to the subject, amount of red blood cells (RBCs) administered to the subject, amount of packed red blood cells (pRBCs) administered to the subject, amount of platelets administered to the subject, level of total packed RBCs, Injury Severity Score (ISS), Abbreviated Injury Scale (AIS) of head, AIS of abdomen, AIS of chest (thorax), Acute Physiology and Chronic Health Evaluation II (APACHE II) score, presence of critical colonization (CC) in a sample from the subject, presence of traumatic brain injury, severity of traumatic brain injury, length of hospital stay, length of intensive care unit (ICU) stay, number of days on a ventilator, disposition from hospital, development of nosocomial infections, level of interferon gamma induced protein 10 (IP-10) in a sample from the subject, level of soluble interleukin 2 receptor (IL-2R) in a sample from the subject, level of IL-2R in a serum sample from the subject, level of interleukin-10 (IL-10) in a sample from the subject, level of interleukin-3 (IL-3) in a sample from the subject, level of interleukin-6 (IL-6) in a sample from the subject, level of interleukin-7 (IL-7) in a sample from the subject, level of interleukin-8 (IL-8) in a sample from the subject, level of monocyte chemoattractant protein 1 (MCP-1) in a sample from the subject, level of monokine induced by gamma interferon (MIG) in a sample from the subject, and level of eotaxin in a sample from the subject.

The clinical parameters may include one or more biological effectors and/or one or more non-biological effectors. As used herein, the term “biological effector” or “biomarker” is used to mean a molecule, such as but not limited to, a protein, peptide, a carbohydrate, a fatty acid, a nucleic acid, a glycoprotein, a proteoglycan, etc. that can be assayed. Specific examples of biological effectors can include, cytokines, growth factors, antibodies, hormones, cell surface receptors, cell surface proteins, carbohydrates, etc. More specific examples of biological effectors include interleukins (ILs) such as IL-Iα, IL-1β, IL-1 receptor antagonist (IL-1RA), IL-2, IL-2 receptor (IL-2R), IL-3, IL-4, IL-5, IL-6, IL-7, IL-8, IL-10, IL-12, IL-13, IL-15, IL-17, as well as growth factors such as tumor necrosis factor alpha (TNFα), granulocyte colony stimulating factor (G-CSF), granulocyte macrophage colony stimulating factor (GM-CSF), interferon alpha (IFN-α), interferon gamma (IFN-γ), epithelial growth factor (EGF), basic endothelial growth factor (bEGF), hepatocyte growth factor (HGF), vascular endothelial growth factor (VEGF), and chemokines such as monocyte chemoattractant protein-1 (CCL2/MCP-1), macrophage inflammatory protein-1 alpha (CCL3/MIP-1α), macrophage inflammatory protein-1 beta (CCL4/MIP-1β), CCL5/RANTES, CCL11/eotaxin, monokine induced by gamma interferon (CXCL9/MIG) and interferon gamma-induced protein-10 (CXCL10/IP10). In some embodiments, the biological effectors are soluble. In some embodiments, the biological effectors are membrane-bound such as a cell surface receptor. In some embodiments, the biological effectors are detectable in a fluid sample of a subject such as serum, wound effluent, and/or plasma.

As used herein, the term non-biological effector is a clinical parameter that is generally considered not to be a specific molecule. Although not a specific molecule, a non-biological effector may nonetheless still be quantifiable, either through routine measurements or through measurements that stratify the data being assessed. For example, number or concentrate of red blood cells, white blood cells, platelets, coagulation time, blood oxygen content, etc. would be a non-biological effector component of the risk profile. All of these components are measureable or quantifiable using routine methods and equipment. Other non-biological components include data that may not be readily or routinely quantifiable or that may require a practitioner's judgment or opinion. For example, wound severity may be a component of the risk profile. While there may be published guidance on classifying wound severity, stratifying wound severity and, for example, assigning a numerical value to the severity, still involves observation and, to a certain extent, judgment or opinion. In some instances the quantity or measurement assigned to a non-biological effector could be binary, e.g., “0” if absent or “1” if present. In other instances, the non-biological effector aspect of the risk profile may involve qualitative components that cannot or should not be quantified.

In some embodiments, the mechanism of injury is a clinical parameter. As used herein, the phrase “mechanism of injury” means the manner in which the subject received an injury and may fall into one of three categories: blast, crush, or gunshot wound (GSW). A blast injury is a complex type of physical trauma resulting from direct or indirect exposure to an explosion. Blast injuries may occur, for example, with the detonation of high-order explosives as well as the deflagration of low order explosives. Blast injuries may be compounded when the explosion occurs in a confined space. A crush injury is injury by an object that causes compression of the body. Crush injuries are common following a natural disaster or after some form of trauma from a deliberate attack. A GSW is an injury that occurs when a subject is shot by a bullet or other sort of projectile from a firearm.

Levels of the clinical parameters can be assayed, detected, measured, and/or determined in a sample taken or isolated from a subject. “Sample” and “test sample” are used interchangeably herein.

Examples of test samples or sources of clinical parameters include, but are not limited to, biological fluids and/or tissues isolated from a subject or patient, which can be tested by the methods of the present disclosure described herein, and include but are not limited to whole blood, peripheral blood, serum, plasma, cerebrospinal fluid, wound effluent, urine, amniotic fluid, peritoneal fluid, pleural fluid, lymph fluids, various external secretions of the respiratory, intestinal, and genitourinary tracts, tears, saliva, white blood cells, solid tumors, lymphomas, leukemias, myelomas, and combinations thereof. In particular embodiments, the sample is a serum sample, wound effluent, or a plasma sample.

In some embodiments, the clinical parameters are one or more of biomarkers, administration of blood products, and injury severity scores. In specific embodiments, the clinical parameters of a subject are selected from one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, or 45 of the clinical parameters listed in Table 1.

TABLE 1
BIOMARKERS

| | | Entrez | Refseq Protein |
|---|---|---|---|
| | | Gene | or Uniprot |
| Symbol | Name | Ref # | Ref # |
| EGF | Epidermal growth factor | 1950 | NP_001171601 |
| | | | NP_001171602 |
| | | | NP_001954 |
| | | | NP_001343950 |
| CCL11 | Eotaxin-1 | 6356 | NP_002977 |
| BFGF | Basic fibroblast growth | 2247 | NP_001997 |
| | factor | | |
| G-CSF | Granulocyte colony- | 1140 | NP_000750 |
| | stimulating factor | | NP_001171618 |
| | | | NP_757373 |
| | | | NP_757374 |
| GM-CSF | Granulocyte-macrophage | 1437 | NP_000749 |
| | colony-stimulating factor | | |
| HGF | Hepatocyte growth factor | 3082 | NP_000592 |
| | | | NP_001010931 |
| | | | NP_001010932 |
| | | | NP_001010933 |
| | | | NP_001010934 |
| IFN-A | Interferon alpha | 3439, 3440, | NP_008831 |
| | | 3441, 3442, | NP_000596 |
| | | 3443, 3444, | NP_066546 |
| | | 3445, 3446, | NP_002160 |
| | | 3447, 3448, | NP_066282 |
| | | 3449, 3450, | NP_066401 |
| | | 3451, 3452 | NP_002161 |
| | | | NP_002162 |
| IFN-Γ | Interferon gamma | 3458 | NP_000610 |
| IL-10 | Interleukin 10 | 3586 | NP_000563 |
| IL-12 | Interleukin 12 | 3592 | (UNIPROT) |
| | | 3593 | P29459 |
| | | | P29460 |
| IL-13 | Interleukin 13 | 3596 | NP_002179 |
| | | | NP_001341920 |
| | | | NP_001341921 |
| | | | NP_001341922 |
| IL-15 | Interleukin 15 | 3600 | NP_000576 |
| | | | NP_751915 |
| IL-17 | Interleukin 17 | 3605, 5982, | NP_002181 |
| | | 5983, 5984, | NP_055258 |
| | | 64806 | NP_037410 |
| | | | NP_612141 |
| | | | NP_073626 |
| IL-1A | Interleukin 1 alpha | 3552 | NP_000566 |
| IL-1B | Interleukin 1 beta | 3553 | NP_000567 |
| IL-1RA | Interleukin 1 receptor | 3557 | NP_000568 |
| | antagonist | | NP_001305843 |
| | | | NP_776213 |
| | | | NP_776214 |
| | | | NP_776215 |
| IL-2 | Interleukin 2 | 3558 | NP_000577 |
| IL-2R | Interleukin 2 receptor | 3559, 3560 | (UNIPROT) |
| | | | P01589 |
| | | | P14784 |
| IL-3 | Interleukin 3 | 3562 | NP_000579 |
| IL-4 | Interleukin 4 | 3565 | NP_000580 |
| | | | NP_758858 |
| | | | NP_001341919 |
| IL-5 | Interleukin 5 | 3567 | NP_000870 |
| IL-6 | Interleukin 6 | 3569 | NP_000591 |
| | | | NP_001305024 |
| IL-7 | Interleukin 7 | 3574 | NP_000871 |
| | | | NP_001186815 |
| | | | NP_001186816 |
| | | | NP_001186817 |
| IL-8 | Interleukin 8 | 3576 | NP_000575 |
| | | | NP_001341769 |
| IP-10 | Interferon gamma-induced | 3627 | NP_001556 |
| | protein 10 | | |
| CCL2/ | Monocyte chemoattractant | 6347 | NP_002973 |
| MCP-1 | protein 1 | | |
| CXCL9/ | Monokine induced by | 4283 | NP_002407 |
| MIG | gamma interferon | | |
| CCL3/ | Macrophage inflammatory | 6348 | (UNIPROT) |
| MIP-1A | protein 1 alpha | | P10147 |
| CCL4/ | Macrophage inflammatory | 6351 | (UNIPROT) |
| MIP-1B | protein 1 alpha | | P13236 |
| CCL5/ | Chemokine (c-c motif) | 6352 | NP_001265665 |
| RANTES | ligand 5 | | NP_002976 |
| TNFA | Tumor necrosis factor alpha | 7124 | NP_000585 |
| VEGF | Vascular endothelial | 7422, 5228, | NP_001020537 |
| | growth factor | 7423, 7424, | NP_001193941 |
| | | 2277 | NP_001230662 |
| | | | NP_005420 |
| | | | NP_004460 |

ADMINISTRATION OF BLOOD
Whole blood cells administered
Red blood cells (RBCs) administered
Packed red blood cells (pRBCs) administered
Platelets administered
Summation of total blood products administered
Level of total packed rbcs
INJURY SEVERITY SCORES
ISS
AIS of the abdomen
AIS of the chest
AIS of an extremity
AIS of the face
AIS of the head
AIS of the skin

In particular embodiments of the methods disclosed herein, the clinical parameters are selected from one or more of a presence of CC in a sample from the subject, level of total RBCs administered to the subject, summation of all blood products administered to the subject, level of IL-2R in a serum sample from the subject, and level of MIG in a serum sample from the subject.

As used herein, the term “summation of all blood products administered to the subject” refers to a value reflecting the total amount of blood products administered to the subject. Blood products include but are not limited to whole blood, platelets, red blood cells, packed red blood cells, and serum. In some embodiments, this value reflects the total amount of blood products needed to stabilize the subject following hemorrhage. Stabilization refers to homeostasis achieved in the subject and is defined as either achieving an equilibrium between bleeding or a complete cessation of hemorrhage.

Interleukin-2 receptor (IL-2R) is a heterotrimeric protein that binds IL-2. The alpha chain of IL-2R is encoded by the IL2RA gene (Entrez gene 3559; UniProt: P01589). The beta chain of IL-2R is encoded by the IL2RB gene (Entrez gene 3560; UniProt: P14784). The gamma chain of IL-2R is encoded by the IL2RG gene (Entrez gene 3561; UniProt: P31785). In some embodiments, the IL-2R is soluble IL-2R. In some embodiments, the IL-2R is cell membrane-bound.

Monokine induced by gamma interferon (MIG) is also known as Chemokine (C-X-C motif) ligand 9 (CXCL9) and is a cytokine that is induced by interferon gamma to chemoattract T-cells. MIG is encoded by the CXCL9 gene (Entrez gene 4283; RefSeq protein: NP_002407).

As used herein, the term critical colonization (or “CC”) is a measure of CFU that the subject has in serum and/or tissue for at least one wound when initially examined by the attending physician. For example, if a subject has CFU of 1×10⁵ per ml of serum, or if at least one wound has CFU of 1×10⁵ per mg of tissue, the subject is said to be “positive” for CC. If the total serum CFU or no single wound has CFU of at least 1×10⁵ the subject is said to be “negative” for CC.

Examples of individual clinical parameters for bacteremia (e.g., components of a risk profile for bacteremia) include but are not limited to a presence of CC in a sample from the subject, level of total RBCs administered to the subject, summation of all blood products administered to the subject, level of IL-2R in a serum sample from the subject, level of MIG in a serum sample from the subject, mechanism of injury, a level of IL-3 in a sample from the subject, a level of IL-8 in a sample from the subject, and a level of IL-6 in a sample from the subject.

Interleukin 3 (IL-3) is a cytokine encoded by the IL3 gene (Entrez gene 3562; RefSeq protein: NP_000579).

Interleukin 6 (IL-6) is a pro-inflammatory cytokine and an anti-inflammatory myokine encoded by the IL6 gene (Entrez gene 3569; RefSeq protein: NP_000591, NP_001305024).

Interleukin 8 (IL-8) is a chemokine encoded by the IL8 gene (Entrez gene 3576; RefSeq protein: NP_000575, NP_001341769).

As used herein, the term “AIS” refers to the abbreviated injury scale, a well-known parameter in the art used routinely in clinics to assess severity of wounds or injuries. In some embodiments, an AIS of 1 is a minor injury, an AIS of 2 is a moderate injury, and AIS of 3 is a serious injury, an AIS of 4 is a severe injury, an AIS of 5 is a critical injury, and an AIS of 6 is an unsurvivable injury.

As used herein, the term “ISS” or “ISS score” refers to the injury severity score, a well-known parameter in the art used routinely in clinics to assess severity of wounds or injuries. ISS is a metric for evaluating severity of injury in trauma patients. It is a composite score by which an AIS score is given for each of several categories of body sites (e.g., Head and Neck, Abdomen, Skin, Chest, Extremities, and Face). The three highest site-specific AIS scores are then squared and added together to give the ISS for the patient or subject as a whole. ISS can range from 0 to 75. If an injury is assigned an AIS of 6 (unsurvivable injury), the ISS score is automatically assigned to 75.

As used herein, assessing an injury such as an abdominal injury or head injury, for the purposes of using these clinical parameters in the systems and methods described herein, means the degree or extent of injury, as reflected in an AIS score of 1-6.

### B. Machine Learning Systems and Methods for Predictive Diagnostic Modeling

In various embodiments, systems and methods of the present disclosure can execute machine learning algorithms to perform data mining, pattern recognition, intelligent prediction, and other artificial intelligence procedures, such as for enabling diagnostic predictions based on clinical data. Machine learning algorithms are increasingly being implemented to reveal knowledge structures that may guide decisions in conditions of limited certainty. Using manual techniques, this would not be possible because of the large number of data points involved. However, in order to use machine learning algorithms effectively, a comparison of models implemented by machine learning algorithms may be required in order to get optimal results out of existing data.

Executing these algorithms can improve the performance of diagnostic prediction technology, such as by increasing accuracy, selectivity, and/or specificity of models used to perform the diagnostic predictions, and thus improve decision-making for and delivery of treatments to subjects. While various machine learning algorithms can be used for such purposes, generate a machine learning system with desired performance characteristics can be highly domain-specific, requiring rigorous modeling, testing, and validation to select appropriate algorithms (or combinations thereof) and the parameters modeled with the algorithms to generate the machine learning system.

In some embodiments, machine learning algorithms can be executed to explore data, usually large amounts of data, to extract patterns and/or systematic relationships between variables, and then to validate the findings by applying the detected patterns to new sets of data. The machine learning algorithms can be implemented in three stages: (1) initial exploration, (2) model building or pattern identification with validation/verification, and (3) deployment, such as by applying the model to new data in order to generate predictions. It will be appreciated that there may be overlap in these stages, and the output of various stages may be fed or fed back to other stages to more effectively generate the desired diagnostic prediction system.

The initial exploration stage may include data preparation. Data preparation may include cleaning data, transforming data, selecting subsets of records and—in case of data sets with large numbers of variables (“fields or dimensions”)—performing variable selection operations (e.g., feature selection, parameter selection), to bring the number of variables to a manageable range (depending on the statistical methods which are being considered). The data on which data preparation is performed may be referred to as training data. In many supervised learning problems, variable selection can be important for a variety of reasons including generalization performance, running time requirements and constraints and interpretational issues imposed by the problem itself. Given that the performance of machine learning algorithms can depend strongly on the quality of the training data used to train the algorithms, variable selection and other data preparation operations can be highly significant for ensuring desired performance.

In some embodiments, data preparation can include executing pre-processing operations on the data. For example, an imputation algorithm can be executed to generate values for missing data. Up-sampling and/or predictor rank transformation can be executed (e.g., for variable selection) to accommodate class imbalance and non-normality in the data.

In some embodiments, executing the imputation algorithm includes interpolating or estimating values for the missing data, such as by generating a distribution (e.g., a Gaussian distribution) of available data for a clinical parameter having missing data, and interpolating values for the missing data based on the distribution. For example, rfImpute from the randomForest R package can be used to impute missing data.

Depending on the nature of the analytic problem, this first stage may involve an activity anywhere between a simple choice of straightforward predictors for a regression model, to elaborate exploratory analyses using a wide variety of graphical and statistical methods in order to identify the most relevant variables and determine the complexity and/or the general nature of models that can be taken into account in the next stage.

Variable selection can include executing supervised machine learning algorithms, such as constraint-based algorithms, constrain-based structure learning algorithms, and/or constraint-based local discovery learning algorithms. Variable selection can be executed to identify a subset of variables in the training data which have desired predictive ability relative to a remainder of the variables in the training data, enabling more efficient and accurate predictions using a model generated based on the selected variables. In some embodiments, variable selection is performed using machine learning algorithms from the “bnlearn” R package, including but not limited to the Grow-Shrink (“gs”), Incremental Association Markov Blanket (“iamb”), Fast Incremental Association (“fast.iamb”), Max-Min Parents & Children (“mmpc”), or Semi-Interleaved Hiton-PC (“si.hiton.pc”) algorithms. R is a programming language and software environment for statistical computing. It will be appreciated that various other implementations of such machine learning algorithms (in R or other environments) may be used to perform variable selection and other processes described herein. Variable selection can search for a smaller dimension set of variables that seek to represent the underlying distribution of the full set of variables, which attempts to increase generalizability to other data sets from the same distribution.

In some embodiments, variable selection is performed to search the training data for a subset of variables which are used as nodes of Bayesian networks. A Bayesian network (e.g., belief network, Bayesian belief network) is a probabilistic model representing a set of variables and their conditional dependencies using a directed acyclic graph. For example, in the context of diagnostic prediction, variable selection can be used to select variables from the training data to be used as nodes of the Bayesian network; given values for the nodes for a specific subject, a prediction of a diagnosis for the subject can then be generated.

Machine learning algorithms can include cluster analysis, regression, both linear and non-linear, classification, decision analysis, and time series analysis, among others. Clustering may be defined as the task of discovering groups and structures in the data whose members are in some way or another “similar”, without using known structures in the data.

Classification may be defined as the task of generalizing a known structure to be applied to new data. Classification algorithms can include linear discriminant analysis, classification and regression trees/decision tree learning/random forest modeling, nearest neighbor, support vector machine, logistic regression, generalized linear models, Naive Bayesian classification, and neural networks, among others. In some embodiments, classification algorithms can be used from the train function of the R caret package, including but not limited to linear discriminant analysis (lda), classification and regression trees (cart), k-nearest neighbors (knn), support vector machine (svm), logistic regression (glm), random forest (rf), generalized linear models (glmnet) and/or naïve Bayes (nb).

A random forest model can include a “forest” of a large number of decision trees, such as on the order of 10²-10⁵ decision trees. The number of decision trees may be selected by calculating an out-of-bag error (the mean prediction error on each training sample, using only the trees that did not have the each training sample in their randomly sampled set of training data, as discussed below) for the resulting random forest model. In some embodiments, the number of decision trees used may be several hundred trees, which can improve computational performance of the machine learning systems by reducing the number of calculations needed to execute the random forest model. The two chief draws of the random forest is that it does not require the data to be either normally distribution or transformed and that the algorithm requires little tuning, which is advantageous when updating data sets, and its numerical process includes cross validation precluding the need for post model-building cross validation.

In some embodiments, each random forest decision tree is generated by bootstrap aggregating (“bagging”), where for each decision tree, the training data is randomly sampled with replacement to generate a randomly sampled set of training data, and then the decision tree is trained on the randomly sampled set of training data. In some embodiments, where variable selection is performed prior to generated the random forest model, the training data is sampled based on the reduced set of variables from variable selection (as opposed to sampling based on all variables).

To perform a prediction given values of variables for a subject, each decision tree is traversed using the given values until a decision rule is reached that is followed by terminal nodes (e.g., presence of disease in the subject, no presence of disease in the subject). The outcome from the decision rule followed by the terminal nodes is then used as the outcome for the decision tree. The outcomes across all decision trees in the random forest model are summed to generate a prediction regarding the subject.

Naïve Bayesian algorithms can apply Bayes' theorem to predict outcomes based on values of variables, such as values of the variables identified using variable selection. The model is called “naïve” due to the assumption that each of the variables is independently associated with having bacteremia. While it may be more realistic for there to be a joint probability for the variables when performing predictions, the naïve approach may provide performance characteristics desirable for the diagnostic prediction system being generated. A naïve Bayes model can be trained by calculating a relationship between values of each variable and the corresponding outcome(s) represented in the training data. For example, in a diagnostic prediction system for a particular disease, values of each variable may be associated with the outcomes of whether or not the particular disease is present. In some embodiments, the relationship may be calculated using a normal distribution for the values of the variables, such that the normal distribution can be used to determine a probability that each variable may have a specified value in the case of (1) the disease being present, or (2) the disease not being present. Then, when executing the trained naïve Bayes to predict the presence of the particular disease for a subject, a probability can be calculated, for each value of each variable, that the variable would have that value given that the particular disease is present in the subject; similarly, a probability can be calculated, for each value of each variable, that the variable would have that value given that the particular disease is not present in the subject. The probabilities for each case can be combined, and then compared to generate a prediction as to whether the particular disease is present in the subject.

In some embodiments, a neural network includes a plurality of layers each including one or more nodes, such as a first layer (e.g., an input layer), a second layer (e.g., an output layer), and one or more hidden layers. The neural network can include characteristics such weights and biases associated with computations that can be performed between nodes of layers. For example, a node of the input layer can receive input data, perform a computation on the input data, and output a result of the computation to a hidden layer. The hidden layer may receive outputs from one or more input layer nodes, perform a computation on the received output(s), and output a result to another hidden layer, or to the output layer. The weights and biases can affect the computations performed by each node, and can be manipulated by an algorithm executing the neural network, such as an optimization algorithm being used to train the neural network to match training data.

Regression analysis attempts to find a function which models the data with the least error. Regression analysis can be used for prediction, as the function can be used to predict a value for a dependent variable given value(s) for independent variable(s).

The second stage—model building or pattern identification with validation/verification—can include considering various models and choosing the best one based on predictive performance. Predictive performance can be correspond to explaining the variability in question and producing stable results across samples. This may sound like a simple operation, but in fact, it sometimes involves a very elaborate process. There are a variety of techniques developed to achieve that goal—many of which are based on so-called “competitive evaluation of models”, that is, applying different models to the same data set and then comparing their performance to choose the best model. These techniques—which are often considered the core of predictive machine learning—can include: bagging (voting, averaging), boosting, stacking (stacked generalizations), and meta-learning. Validation can include comparing the output of a selected model to validation data. For example, a portion of the training data can be held separately from that which is used to train the model, and then can be used to confirm the performance characteristics of the trained model.

There are different scenarios in which a comparison of machine learning algorithms (and combinations thereof) may be useful. Many application scenarios do not have single models, but multiple, related ones. Some typical examples are machine learning algorithms trained based on data derived at different points in time or in different subsets of the data, e.g., production quality data from different production sites. Another common case is representing the same data with machine learning algorithms on different types of machine learning algorithms in order to capture different aspects of the data. In all these cases, not only the individual data mining models are of interest, but also similarities and differences between them. Such differences may tell, for instance, how production quality and dependencies develop over time, how machine learning algorithms of different types differ in their ways of representing different products produced at the same facility or, how the production facilities differ between each other.

Machine learning algorithms (and combinations thereof) can be compared using performance metrics. The performance metrics may be selected based on the intended application of the machine learning algorithms (and the predictive models created using the machine learning algorithms). For diagnostic prediction models, the performance metrics can include Kappa score, Accuracy score, sensitivity, specificity, total, positive class, and negative class out-of-bag (OOB) error estimates, receiver operator characteristic curves (ROCs), areas under curve (AUCs), confusion matrices, Vickers and Elkins' Decision Curve Analysis (DCA), or other measures of the performance of the machine learning algorithms.

The Kappa score represents a comparison of an observed accuracy of the diagnostic prediction model to an expected accuracy. For example, the Kappa score measures how closely the diagnostic prediction model matches training data (e.g., the relationships between variables and the corresponding outcomes known in the training data), controlling for the accuracy of a random classifier as measured by the expected accuracy.

The sensitivity measures a proportion of positive results from the prediction that are correctly identified as such. As such, the sensitivity can quantify the avoidance of false negatives. The specificity measures a proportion of negative results from the prediction that are correctly identified as such.

OOB measures prediction error in random forest and other machine learning techniques that rely on bootstrapping to sub-sample training data. The OOB error analysis can be used to show how the variable selected models can improve the OOB error (predictive performance) for the positive class.

The ROC curve is a plot of true positive rate (sensitivity) as a function of false positive rate (specificity). The AUC represents the area under the ROC curve. For example, model performance can be further assessed using the plot.roc in R command to compute the Receiver Operator Characteristic Curves (ROC) and area under curve (AUC). Decision Curve Analysis (DCA) can be used to calculate the net benefit of treatment based on the diagnoses predicted by the diagnostic prediction models, as compared to baseline treatment methodologies such as assuming that all patients are test positive and therefore treating everyone, or assuming that all patients are test negative and therefore offering treatment to no one. The DCA curve plots the net benefit of the diagnostic prediction model as a function of a threshold probability, the threshold probability being a value at which the subject would opt for treatment given the relative harms of false positive predictions. DCA is used to compare various predictive and diagnostic paradigms in terms of net benefit to the patient. A typical DCA analysis will compare the null model, treat no one, to various alternative models, such as “treat-all” or treat according to the guidance of models built on biomarker predictors. DCA analysis can be interpreted as showing positive net-benefit to the patient if the decision curve for a particular model is above the null model (x axis), and to the right of the “treat-all” model. Net-benefit is defined mathematically as a summation of model performance (for instance propensity to predict false positive or false negatives) over a series of predictive threshold cutoffs and the respective sensitivity/specificity at those thresholds. The threshold cutoffs could be thought of as the point at which a decision to treat would be made given the relative harms and benefits of treating given the uncertainty of the prediction at that threshold. This analysis demonstrates the threshold cutoffs where the predictive models are most useful to the patient. The dca R command from the Memorial Sloan Kettering Cancer Center website, www.mskcc.org, can be used to compute the Decision Curve Analysis (DCA).

The third stage—deployment—can include using the model selected as best in the previous stage and applying it to new data in order to generate predictions or estimates of the expected outcome. For example, the selected model can be executed using clinical data specific to a particular subject in order to predict the expected outcome for the particular subject. In some embodiments, the clinical data for the particular subject can be used to update the model, particularly after confirming whether or not the disease is present in the particular subject.

### C. Systems and Methods for Subject-Specific Using Predictive Modeling to Predict Bacteremia Outcomes

In some embodiments, the systems and methods described herein for generating predictive models for predicting subject-specific bacteremia outcomes involve the execution of two main steps: variable selection and binary classification. An advantage of variable selection is that variable selection can search for a smaller dimension set of variables that seek to represent the underlying distribution of the full set of variables, which attempts to increase generalizability to other data sets from the same distribution. In some embodiments, such as where the datasets are relatively small, computational time may not be a consideration. Since variable selection is based on a better representation of the underlying distribution of the full variables set, in theory, they should be more generalizable and less susceptible to over fitting.

In building machine learning solutions to predict clinical outcomes, it is typically unfeasible to provide the machine learning algorithms with an exhaustive list of clinical parameters which may be relevant to the clinical outcomes being predicted. For example, with very large lists of clinical parameters, there may be significant noise, highly correlated variables, and other opportunities for introducing errors which can adversely affect the ability of the machine learning algorithms to generate predictive models (by performing variable selection and classification) which meet desired performance metrics.

In some situations, the number of clinical parameters may be on the order of 5000-50000 variables, from which the machine learning solutions will have to perform variable selection and other operations. To do so would require incredibly large computing resources which are not readily available, making such processes virtually impossible. Additionally, even if such resources were available, the opportunities for introducing error in the resulting solutions would counteract any added benefit from considering all variables.

In the present solution, over 7000 initial clinical and nonclinical parameters were available regarding the subjects that could potentially be used to train the machine learning solutions. These clinical parameters fell into a wide variety of categories, such as demographics, wound type, wound mechanism, wound location, fracture characteristics, administration of blood products, injury severity scores, treatment(s), tobacco usage, activity levels, surgical history, nutrition, serum protein expression, wound effluent protein expression, tissue bacteriology, mRNA expression, and Raman spectroscopy. From these categories, using expert knowledge, the following were selected for usage with the machine learning solutions disclosed herein: serum protein expression, administration of blood products, and injury severity scores. The expert selection process was important for distilling the many possible parameters to the minimum number that will result in the strongest predictive power. This process can also improve the methods of the present solution described in Section D below.

In some embodiments, clinical parameters that fall within the biomarkers category include level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-Iα) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, or level of vascular endothelial growth factor (VEGF) in a sample from the subject, among others.

In some embodiments, clinical parameters that fall within the administration of blood products category include amount of whole blood cells administered to the subject, amount of red blood cells (RBCs) administered to the subject, amount of packed red blood cells (pRBCs) administered to the subject, amount of platelets administered to the subject, summation of all blood products administered to the subject, or level of total packed RBCs, among others.

In some embodiments, clinical parameters that fall within the injury severity scores category include Injury Severity Score (ISS), Abbreviated injury scale (AIS) of abdomen, AIS of chest (thorax), AIS of extremity, AIS of face, AIS of head, or AIS of skin, among others.

The machine learning solutions described herein can execute variable selection on the clinical parameters within the identified categories to generate predictive models for predicting bacteremia outcomes.

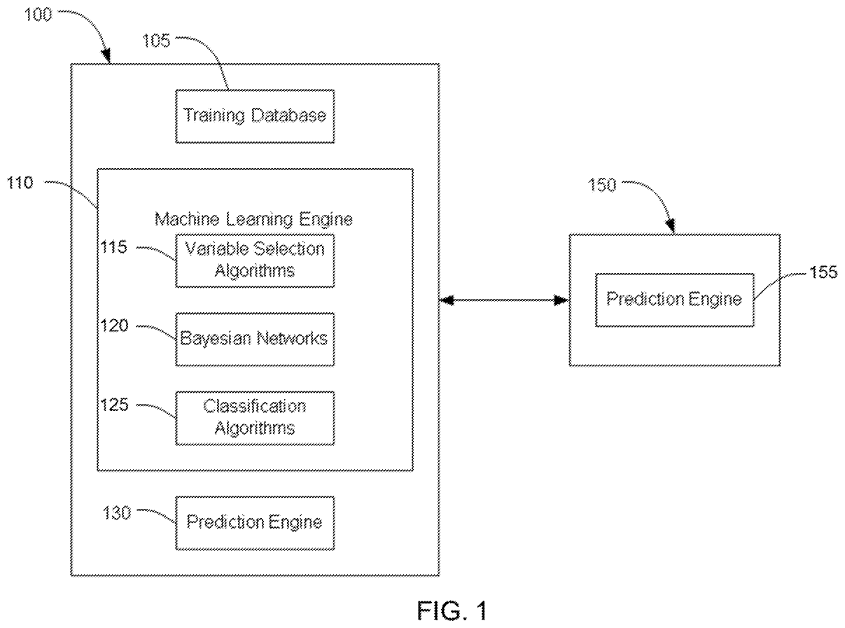

Referring now to FIG. 1, a clinical outcome prediction system (COPS) 100 is shown according to an embodiment of the present disclosure. The COPS 100 includes a training database 105, a machine learning engine 110, and a prediction engine 130. The COPS 100 can be implemented using features of the computing environment described below in Section D. For example, the COPS 100 can include or be coupled to display device(s) to display output from the COPS 100, such as to display predictions of risks of bacteremia in subjects.

The COPS 100 can execute various machine learning processes, including but not limited to those described above in Section B. The COPS 100 can be implemented using a computer including a processor, where the computer is configured or programmed to generate outputs including one or more predictions of bacteremia outcomes, risk profiles, and/or to determine statistical risk. The COPS 100 can display the outputs on a screen that is communicatively coupled to the computer. In some embodiments, two different computers can be used: a first computer configured or programmed to generate risk profiles and a second computer configured or programmed to determine statistical risk. Each of these separate computers can be communicatively linked to its own display or to the same display.

### Training Database

The training database 105 stores values of clinical parameters associated with bacteremia outcomes in subjects. The values of the clinical parameters can be received and stored for each of a plurality of first subjects. The first subjects may have an injury, condition, or wound that puts the subject at risk of developing bacteremia, such as discussed above. The training database 105 can receive and store first values of at least one clinical parameter of a plurality of clinical parameters and a corresponding bacteremia outcome. The training database 105 can associate the first values of the plurality of clinical parameters to the corresponding bacteremia outcome for each of the plurality of first subjects. In some embodiments, the training database 105 stores first values of the plurality of clinical parameters that are associated, for each subject, with a single point in time.

The clinical parameters can include gender, age, date of injury, location of injury, presence of abdominal injury, mechanism of injury, wound depth, wound surface area, number of wound debridements, associated injuries, type of wound closure, success of wound closure, requirement for transfusion, total number of blood products transfused, amount of whole blood cells administered to the subject, amount of RBCs administered to the subject, amount of pRBCs administered to the subject, amount of platelets administered to the subject, level of total pRBCS, Injury Severity Score (ISS), AIS of head, AIS of abdomen, AIS of chest (thorax), Acute Physiology and Chronic Health Evaluation II (APACHE II) score, presence of critical colonization (CC) in a sample from the subject, presence of traumatic brain injury, severity of traumatic brain injury, length of hospital stay, length of intensive care unit (ICU) stay, number of days on a ventilator, disposition from hospital, development of nosocomial infections, level of interferon gamma induced protein 10 (IP-10) in a sample from the subject, level of soluble interleukin 2 receptor (IL2R), in a sample from the subject, level of interleukin-10 (IL-10) in a sample from the subject, level of interleukin-3 (IL-3) in a sample from the subject, level of interleukin-6 (IL-6) in a sample from the subject, level of interleukin-7 (IL-7) in a sample from the subject, level of interleukin-8 (IL-8) in a sample from the subject, level of monocyte chemoattractant protein 1 (MCP-1) in a sample from the subject, level of monokine induced by gamma interferon (MIG) in a sample from the subject, and level of eotaxin in a sample from the subject. The clinical parameters can include at least one selected from Luminex proteomic data, RNAseq, transcriptomic data, quantitative polymerase chain reaction (qPCR) data, and quantitative bacteriology data.

The bacteremia outcome can be based on presence of bacteria in the blood such as may be diagnosed through isolation of a pathogen from at least one quantitated blood culture. In some embodiments, a pathogen is isolated from at least two blood cultures. The bacteremia outcome may be a binary variable (e.g., bacteremia is present in the first subject or bacteremia is not present in the first subject).

The COPS 100 can execute pre-processing on the data stored in the training database 105. Pre-processing may be performed before variable selection and/or classification are performed on the data. In some embodiments, COPS 100 can execute an imputation algorithm to generate values for missing data in the training database 105. The training database 105 may include values of clinical parameters from disparate sources, which may be inconsistent. For example, the training database 105 may include values for IL-10 but not IL-3 for one subject, and values for IL-3 but not IL-10 for another subject. The COPS 100 can execute the imputation algorithm to impute values for IL-3 for the one subject and for IL-10 for the other subject to generate values for the missing data. For example, rfImpute from the randomForest R package can be used to impute missing data.

In some embodiments, the COPS 100 executes at least one of up-sampling or predictor rank transformations on the data of the training database 105. Up-sampling and/or predictor rank transformation can be executed only for variable selection to accommodate class imbalance and non-normality in the data.

### Machine Learning Engine

The machine learning engine 110 can generate models for predicting bacteremia outcomes (and risks thereof) which use a reduced set of clinical parameters as variables. The machine learning engine 110 can execute variable selection (e.g., feature selection, parameter selection) to select a subset of model parameters from the plurality of clinical parameters. The variable selection can be used to identify biological effector and non-biological effector components that are critical to the risk profiles (e.g., bacteremia outcomes or associated risks thereof) stored in the training database 105. The machine learning engine 110 can execute classification on the selected model parameters to select a candidate model for generating bacteremia outcome/risk predictions.

In some embodiments, the machine learning engine 110 executes a plurality of variable selection algorithms 115 using the training database 105 to select a subset of model parameters for each variable selection algorithm 115. The subsets of model parameters are selected from the plurality of clinical parameters of the training database 105, such that a count of each subset of model parameters is less than a count of the clinical parameters.

In some embodiments, the variable selection algorithms 115 executed by the machine learning engine 110 include supervised machine learning algorithms. The machine learning algorithms can be constraint-based algorithms, constraint-based structure learning algorithms, and/or constraint-based local discovery learning algorithms. For example, the machine learning engine 110 can execute machine learning algorithms from the “bnlearn” R package, including but not limited to the Grow-Shrink (“gs”), Incremental Association Markov Blanket (“iamb”), Fast Incremental Association (“fast. iamb”), Max-Min Parents & Children (“mmpc”), or Semi-Interleaved Hiton-PC (“si.hiton.pc”) algorithms.

For example, the machine learning engine 110 can execute the variable selection algorithms 115 to perform variable selection on the entire set of serum Luminex variables as well as available clinical variables, using constraint-based algorithms and constraint-based local discovery learning algorithms from the “bnlearn” R package to search the input dataset for nodes of Bayesian networks. In some embodiments, the training database 105 includes summations of wound volume and wound surface area to account for patient wound burden.

For each variable selection algorithm 115, the machine learning engine 110 uses the corresponding subset of model parameters (selected from the plurality of clinical parameters) as nodes of a Bayesian network. The machine learning engine 110 generates each Bayesian network to represent conditional dependencies between the subset of model parameters and the corresponding bacteremia outcomes stored in the training database 105. As such, the machine learning engine 110 can select the nodes as the reduced variable sets represented by the subset of model parameters selected by each variable selection algorithm 115.

In some embodiments, prior to performing variable selection (and classification) on the clinical parameters of the training database 105, the machine learning engine 110 can randomly re-order the plurality of clinical parameters.

The machine learning engine 110 can execute classification algorithms 125 (e.g., binary classification algorithms) for each subset of model parameters to generate predictions of bacteremia outcomes based on the subsets of model parameters. In some embodiments, the machine learning engine 110 executes classification algorithms 125 including but not limited to linear discriminant analysis (lda), classification and regression trees (cart), k-nearest neighbors (knn), support vector machine (svm), logistic regression (glm), random forest (rf), generalized linear models (glmnet) and/or naïve Bayes (nb). The classification algorithms 125 may be retrieved from the train function of the R caret package. The classification algorithms 125 may be executed by identifying first values of clinical parameters in the training database 105 corresponding to each subset of model parameters, and generating predictions of bacteremia outcomes using the identified first values.

Executing a random forest model classification algorithm 125 can include generating a plurality of decision trees using the training database 105. In various embodiments, a count of the decision trees can be greater than 1000 (e.g., 10001 decision trees). Each decision tree may be generated by bootstrap aggregating with replacement the first values of the plurality of clinical parameters in the training database 105. The decision trees may be generated to make decisions using the subset of model parameters.

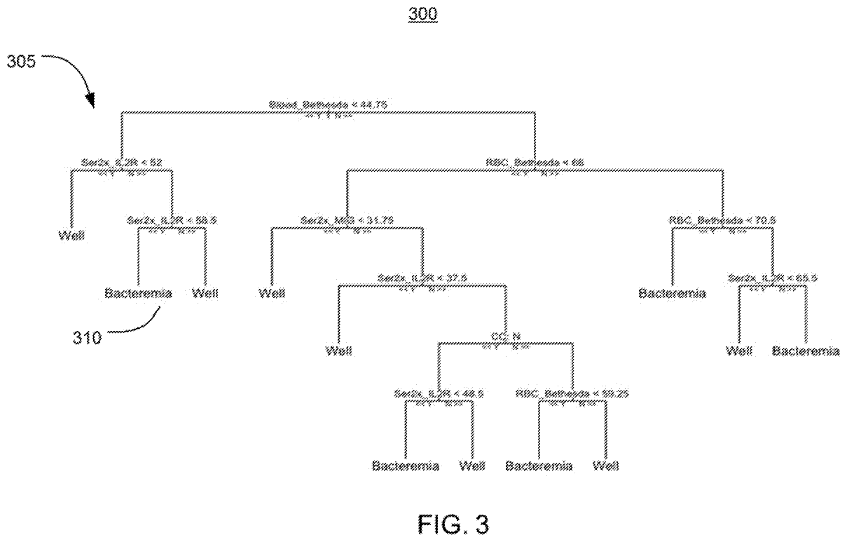

To generate predictions of bacteremia outcomes, the machine learning engine 110 can use test values for the model parameters as inputs in the random forest model classification algorithm 125. For example, referring to FIG. 3, an embodiment of a decision tree 300 which can be generated by the machine learning engine 100 using the training database 105 is shown. The decision tree 300 includes a hierarchical organization of nodes 305, including terminal nodes 310 where, based on the decision made, the decision tree 300 can output a prediction of a bacteremia outcome (e.g., an indication that the subject has bacteremia or that the subject is well). Decisions are made by the decision tree 300 based on a subset of model parameters including amount of blood administered to the subject, amount of RBCs administered to the subject, IL2R, MIG, and CC. For example, given a subject having the following values for the model parameters: amount of blood administered to the subject=22, IL2R=55, RBCs amount of administered to the subject=65, MIG=30, and CC=Yes, the random forest model classification algorithm 125 can traverse the decision tree 300 as follows: at the first node, the amount of blood administered to the subject (22) is determined to be less than 44.75 (resulting in a “Yes” output); next, the IL2R (55) is determined to not be less than 52 (resulting in a “No” output); finally, at the terminal node 310, the IL2R (55) is determined to be less than 58.5, resulting in a “Bacteremia” output. The random forest model classification algorithm 125 can count the number of bacteremia outcomes (e.g., Bacteremia vs. Well) calculated by each decision tree 300, and output the predicted bacteremia outcomes based on the counts. For example, the random forest model classification algorithm 125 can compare the count of “Bacteremia” outputs to the count of “Well” outputs and output the predicted bacteremia outcome to indicate that the subject is predicted to have bacteremia responsive to the count of Bacteremia outputs being greater than the count of Well outputs (or vice versa). The random forest classification algorithm 125 can also output the prediction of the bacteremia outcome as a probability based on the number of Bacteremia outcomes: for example, if the random forest model includes 10000 decision trees, of which 5000 indicate a Bacteremia outcome, the random forest model classification algorithm 125 can output the prediction of bacteremia outcome as a probability of 50%.

Referring back to FIG. 1, the machine learning engine 110 uses the predictions of bacteremia outcomes to calculate performance metrics. For example, the machine learning engine 110 can calculate a performance metric for each combination of (i) a subset of model parameters (selected by each variable algorithm 115) and (ii) a classification algorithm 125 used to generate the predictions of bacteremia outcomes. The performance metrics can represent the ability of each combination to predict bacteremia outcomes.

The machine learning engine 110 can calculate a performance metric including at least one of a Kappa score, a sensitivity, or a specificity. The Kappa score indicates a comparison of an observed accuracy of the combination of the subset of model parameters and the classification algorithm to an expected accuracy. In some embodiments, the machine learning engine 110 can generate an ROC curve based on the sensitivity and the specificity.

The machine learning engine 110 can also calculate an AUC based on the ROC curve. In some embodiments, the candidate classification algorithm 125 can be evaluated by further performance metrics. For example, the candidate classification algorithm 125 can be evaluated based on Accuracy, No Information Rate, positive predictive value and negative predictive value.

The machine learning 110 can apply various policies, heuristics, or other rules based on the performance metric(s) to select a candidate classification algorithm 125 (and corresponding subset of model parameters selected by one of the variable selection algorithms 115). For example, values for each performance metric can be compared to respective threshold values, and a classification algorithm 125 can be determined to be a candidate classification algorithm 125 (or a potential candidate) responsive to the value for the performance metric exceeding the threshold. The machine learning engine 110 can assign weights to each performance metric to calculate a composite performance metric. The machine learning engine 110 can evaluate performance metrics in a specified order.

In some embodiments, the machine learning engine 110 selects the candidate classification algorithm 125 and corresponding subset of model parameters based on the rule: identify the combination having (1) a highest Kappa score; subsequently, (2) a highest sensitivity; and (3) subsequently, a specificity greater than a threshold specificity.

The machine learning engine 110 can execute decision curve analysis (DCA) to evaluate the performance of the candidate classification algorithm 125 and/or with confusion matrices. DCA can be used to assess the net benefit of using the candidate classification algorithm 125 in a clinical setting as compared to a null model, a treat no one paradigm, or a “treat-all” intervention paradigm. The DCA can be executed to validate the performance of the candidate classification algorithm 125, and/or to select the candidate classification algorithm 125 from amongst several classification algorithms 125 having similar performance under other performance metrics.

The machine learning engine 110 can be executed in multiple iterations. For example, the data of the training database 105 can be run through the variable selection and binary classification algorithms more than once, for example, 10, 20, 30, 40, 50 or even more times.

In some embodiments, the candidate model (combination of subset of model parameters and candidate classification algorithm 125) generated by the machine learning engine 110 can be compared in performance to a model generated using the full set of clinical parameters of the training database 105. For example, the machine learning engine 110 can execute a classification algorithm 125 using the full set of clinical parameters, in a similar manner as for executing the classification algorithms 125 based on the subsets of model parameters, to represent a baseline for model performance. The candidate model can be compared to the model generated using the full set of clinical parameters using DCA. The machine learning engine 110 can execute an imputation algorithm to process clinical parameters with missing data.

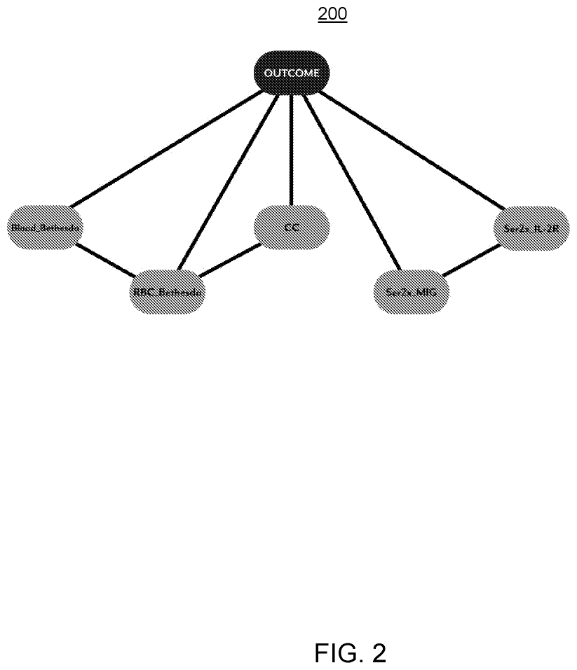

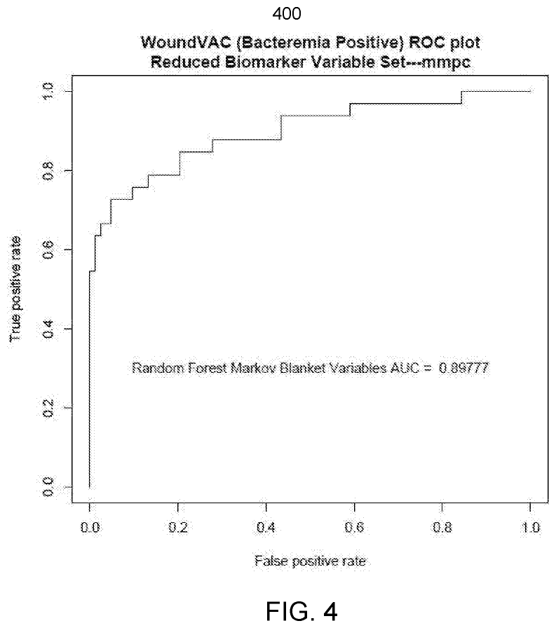

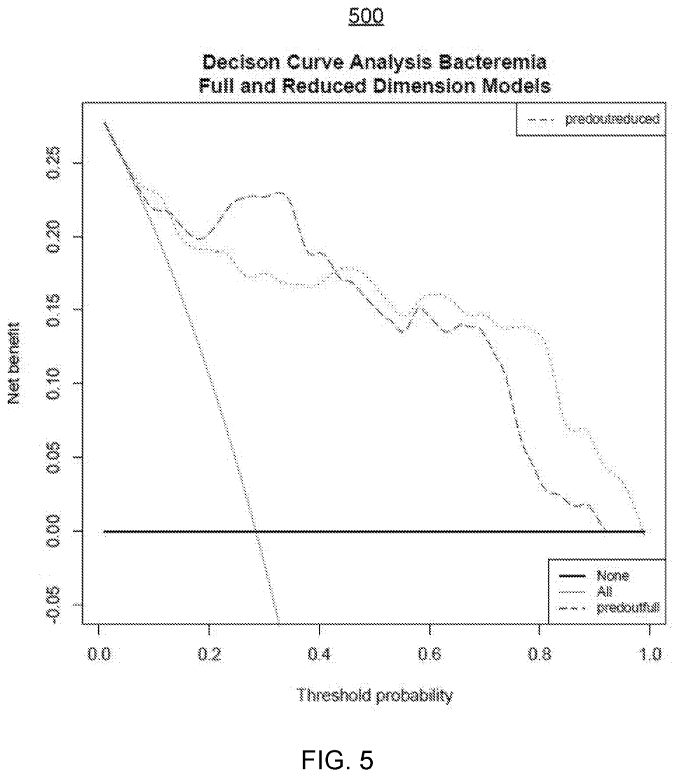

Referring now to FIGS. 2-5, model parameters and performance metrics of a candidate classification algorithm executed using the selected model parameters are illustrated. Briefly, FIG. 2 illustrates a Bayesian network 200 based on the subset of model parameters; FIG. 3 illustrates the decision tree 300 as discussed above, FIG. 4 illustrates an ROC curve, along with the associated AUC, sensitivity (true positive rate), and specificity (false positive rate) for the candidate classification algorithm; and FIG. 5 illustrates a DCA performed on the candidate classification algorithm.

Referring further to FIG. 2, in the illustrated example, the machine learning engine 110 can perform variable selection using a plurality of variable selection algorithms 115 to generate a Bayesian network 200. The machine learning engine 110 can calculate performance metrics to determine which subset of model parameters should be used for predicting bacteremia outcomes. For example, the machine learning engine 110 can determine that the subset of model parameters selected by the max-min parents and children (MMPC) algorithm run in the random forest binary classification algorithm 125 outperform all other subsets of model parameters with all other binary classification algorithms 125. In the illustrated embodiment, the subset of model parameters include the following clinical parameters: blood administered to the subject, RBCs administered to the subject, CC, IL-2R, and MIG.

Referring further to FIG. 4, a chart 400 of performance metrics of the candidate classification algorithm 125 is illustrated. As shown in the chart 400, the machine learning engine 110 can calculate the performance metrics for the candidate classification algorithm 125 using the above subset of model parameters to include an AUC of 0.89777.

Comparisons of the candidate classification algorithm 125 to the full variable models can demonstrate better performance in the candidate classification algorithm 125. This is a key strength of the systems and methods described herein, as over-parameterization frequently leads to model underperformance. In the illustrated embodiment, the candidate classification algorithm 125, the ROC curves and their respective AUCs demonstrated good predictive ability. Similarly, candidate classification algorithm 125 had higher Accuracy and Kappa statistics than the full variable models.

In some embodiments, the COPS 100 can increase the computational performance of a computer system (e.g., processing speed, memory usage) by using the subset of model parameters relative to the full set of clinical parameters to generate predictions of bacteremia outcomes. For example, the COPS 100 can execute fewer calculations to generate each bacteremia outcome prediction, yet avoid over parametrization and other model performance issues by using the subset of model parameters.

Referring further to FIG. 5, a DCA 500 is shown based on the candidate classification algorithm 125. The candidate classification algorithm 125 can demonstrate superior performance based on DCA: for the vast majority of threshold probabilities for net benefit of treatment, the candidate classification algorithm 125 demonstrated greater net benefit than the full variable model as well as treat-all and treat-none paradigms.

### Prediction Engine

Referring back to FIG. 1 and further to FIG. 3, in some embodiments, the COPS 100 includes a prediction engine 130. The prediction engine 130 can predict a bacteremia outcome specific to at least one second subject. The at least one second subject may have an injury. The prediction engine 130 can receive, for the at least one second subject, a second value of at least one clinical parameter of the plurality of clinical parameters.

In some embodiments, at least one of the received second values corresponds to a model parameter of the subset of model parameters used in the candidate classification algorithm 125. If the prediction engine 130 receives several second values of clinical parameters, of which at least one does not correspond to a model parameter of the subset of model parameters, the prediction engine 130 may execute an imputation algorithm to generate a value for such a missing parameter.

The prediction engine 130 can execute the selected candidate classification algorithm 125 using the corresponding subset of model parameters and the second value of the at least one clinical parameter to calculate the bacteremia outcome specific to the at least one second subject. In an example, the selected candidate classification algorithm 125 may include a random forest model based on the following model parameters (and received the indicated second values for the second subject): amount of blood administered to the subject=22, IL2R=55, RBCs amount of administered to the subject=65, MIG=30, and CC=Yes. As described above, the selected candidate classification algorithm 125 can output a prediction indicative of the second subject having bacteremia (or being at a higher risk for bacteremia relative to a reference risk, or being likely to have bacteremia).

As shown in FIG. 1, the COPS 100 includes the prediction engine 130. In some embodiments, a remote device 150 may additionally or alternatively include a prediction engine 155. The prediction engine 155 can incorporate features of the prediction engine 130. The remote device 150 can incorporate features of the computing environment described in Section D below, such as by being implemented as a portable electronic device. The remote device 150 can communicate with the COPS 100 using any of a variety of wired or wireless communication protocols (including communicating via an Internet protocol system or other intermediary communication system). For example, the remote device 150 can receive the prediction engine 130 (or the candidate classification algorithm 125 with the corresponding subset of model parameters) from the COPS 100.

In various embodiments, the COPS 100 and/or the remote device 150 can receive the second values of the plurality of clinical parameters through a user interface, and can output the predictions of bacteremia outcomes responsive to receiving the second values. The remote device 150 can be implemented as a client device executing the prediction engine 155 as a local application which receives the second values and transmits the second values to the COPS 100; the COPS 100 can be implemented as a server device which calculates the prediction of the bacteremia outcome specific to the second subject and transmits the calculated prediction to the prediction engine 155. The remote device 150 may then output the calculated prediction received from the COPS 100.

In some embodiments, the COPS 100 can update the training database 105 based on the second values received for the second subjects, as well as the predicted bacteremia outcomes. As such, the COPS 105 can continually learn from new data regarding subjects. The COPS 100 can store the predicted bacteremia outcome with an association to the second value(s) received for the second subject in the training database 105. The predicted bacteremia outcome may be stored with an indication of being a predicted value (as compared to the known bacteremia outcomes for the plurality of first subjects), which can enable the machine learning engine 110 to process predicted outcome data stored in the training database 105 differently than known outcome data. In addition, it will be appreciated that over time, the second subject based on which a predicted outcome was generate may also have a known bacteremia outcome (e.g., based on the onset of symptoms indicating that the second subject has bacteremia, or based on an indication that the second subject does not have bacteremia, such as a sufficient period of time passing subsequent to the generation of the predicted bacteremia outcome). The COPS 100 can store the known bacteremia outcome with an association to the second value(s) received for the second subject. The COPS 100 can also store the known bacteremia outcome with an indication of an update relative to the predicted bacteremia outcome, which can enable the machine learning engine 110 to learn from the update and thus improve the variable selection and classification processes used to generate and select the candidate classification algorithm/subset of model parameters for use by the prediction engine 130. In some embodiments, the COPS 100 calculates a difference between the predicted bacteremia outcome and the known bacteremia outcome, and stores this difference as the indication of the update.

Referring now to FIG. 5, a method 500 for predicting subject-specific bacteremia outcomes is illustrated according to an embodiment of the present disclosure. The method 500 can be performed by various systems described herein, including the COPS 100 and/or the remote device 150.

At 505, first values of clinical parameters and corresponding bacteremia outcomes for a subject are received. The first subject may have an injury. In some embodiments, the first values of the plurality of clinical parameters that are associated, for each subject, with a single point in time. At 510, a training database is generated associating the first values to the corresponding bacteremia outcomes.

In some embodiments, pre-processing is executed on the data stored in the training database. Pre-processing may be performed before variable selection and/or classification are performed on the data. In some embodiments, an imputation algorithm can be executed to generate values for missing data in the training database 105. In some embodiments, at least one of up-sampling or predictor rank transformations is executed on the data of the training database. Up-sampling and/or predictor rank transformation can be executed only for variable selection to accommodate class imbalance and non-normality in the data.

At 515, a plurality of variable selection algorithms are executed using the data stored in the training database to select, for each variable selection algorithm. The subsets of model parameters are selected from the plurality of clinical parameters of the training database, such that a count of each subset of model parameters is less than a count of the clinical parameters. Variable selection algorithms such as constraint-based algorithms, constrain-based structure learning algorithms, and/or constraint-based local discovery learning algorithms can be used to select the subsets of model parameters. The subsets of models parameters can be used as nodes of Bayesian networks, such that the model parameters represent conditional dependencies between the plurality of model parameters and the corresponding bacteremia outcomes stored in the training database. In some embodiments, the clinical parameters are randomly re-ordered prior to variable selection.

At 520, at least one classification algorithm is executed using each subset of model parameters to generate predictions of bacteremia outcomes based on the subsets of model parameters. The classification algorithms may be executed by identifying first values of clinical parameters in the training database corresponding to each subset of model parameters, and generating predictions of bacteremia outcomes using the identified first values. In some embodiments, the classification algorithms include a plurality of linear discriminant analysis (lda), classification and regression trees (cart), k-nearest neighbors (knn), support vector machine (svm), logistic regression (glm), random forest (rf), generalized linear models (glmnet) and/or naïve Bayes (nb) algorithms.

Executing a random forest model classification algorithm includes generating a plurality of decision trees using the training database (e.g., using bootstrap aggregation), and executing each decision tree to generate bacteremia outcomes which can be combined to calculate a predicted bacteremia outcome.

At 525, at least one performance metric is calculated for each classification algorithm (e.g., each combination of (i) a subset of model parameters selected using a variable selection algorithm and (ii) a classification algorithm used to generate bacteremia outcome predictions). The performance metrics can represent the ability of each combination to predict bacteremia outcomes.

The performance metric can include at least one of a Kappa score, a sensitivity, or a specificity. The Kappa score indicates a comparison of an observed accuracy of the combination of the subset of model parameters and the classification algorithm to an expected accuracy. In some embodiments, an ROC curve can be generated based on the sensitivity and the specificity. An AUC can be calculated based on the ROC curve. In some embodiments, the candidate classification algorithm can be evaluated by further performance metrics. For example, the candidate classification algorithm can be evaluated based on Accuracy, No Information Rate, positive predictive value and negative predictive value.

At 530, a candidate classification algorithm is selected based on the performance metric(s). Various policies, heuristics, or other rules can be applied based on the performance metric(s) to select a candidate classification algorithm (and corresponding subset of model parameters selected by one of the variable selection algorithms). For example, values for each performance metrics can be compared to respective threshold values, and a classification algorithm can be determined to be a candidate classification algorithm (or a potential candidate) responsive to the value for the performance metric exceeding the threshold. In some embodiments, the candidate classification algorithm and corresponding subset of model parameters are selected based on the rule: identify the combination having (1) a highest Kappa score; subsequently, (2) a highest sensitivity; and (3) subsequently, a specificity greater than a threshold specificity.

At 535, second values of clinical parameters are received. The second values may be received for at least one second subject having an injury. In some embodiments, at least one of the received second values corresponds to a model parameter of the subset of model parameters used in the candidate classification algorithm. If several second values of clinical parameters are received, of which at least one does not correspond to a model parameter of the subset of model parameters, an imputation algorithm may be executed to generate a value for such a missing parameter.

At 540, the candidate classification algorithm is executed using the corresponding subset of model parameters and the second value of the at least one clinical parameter to calculate the prediction of the bacteremia outcome specific to the at least one second subject.

At 545, the predicted bacteremia outcome specific to the at least one second subject is outputted. For example, the predicted bacteremia outcome may be displayed on an electronic device to a user, or may be provided as an audio output. The predicted bacteremia outcome may be transmitted to another device. The predicted bacteremia outcome may include at least one of an indication that the second subject has bacteremia, that the second subject is likely to have bacteremia (e.g., relative to a confidence threshold), or that the second subject has an increased risk for bacteremia relative to a reference risk level.

In some embodiments, the methods described herein involve two main steps: variable reduction and binary classification. To perform variable selection on the entire set of serum and effluent variables as well as available clinical variables a “bnlearn” R package(1) can be employed. Variable selection may be performed by removing variables that are highly correlated. Several algorithms can be used to search the input dataset with ranked predictors to find a reduced variable set that best represented the underlying distribution of all variables with respect to the infectious complication outcomes. A feature selection filter algorithm can be used to choose the reduced variable set, such as one or more of the inter.iamb, fast.iamb, iamb, gs, mmpc, and si.hiton.pc algorithms. While all algorithms can examined for testing, selected algorithms may be used to choose the nodes of the corresponding Bayesian network as the reduced variable set. For example, in some embodiments, one or more of the Maximum Minimum Parents Children (mmpc) and/or the inter.iamb algorithm can be used to choose the nodes of the corresponding Bayesian network as the reduced variable set. Once each algorithm selected variables for inclusion, a Bayesian network can be constructed, and the quality of statistical models can be compared using the Bayesian Information Criterion (BIC). The models with the largest BIC can be flagged for further modeling and analysis.

Next, a random forest model can be constructed using the full set of variables pulled from the raw data as a baseline. To handle process samples with missing data, R packages rfImpute can be used (for example). The total, positive class and negative class out-of-bag (OOB) error estimates of the model can be plotted and then the Accuracy and Kappa scores can be calculated, such as by using the “randomForest” R package. (This full set of variables can be the same full set from which variables were selected.) Next, a random forest model can be constructed with the Bayesian network-selected variables or by removing variables that are highly correlated with those that are used. Optionally, the random forest performance with OOB error plots, Accuracy and Kappa scores can be assessed. The model with the smallest OOB errors and BIC scores and the highest Accuracy and Kappa scores can be chosen. Both random forest models can be constructed using a plurality of classification and regression trees and square root of p variables randomly sampled as candidates at each split, where p is the number of variables in the model. The number of classification and regression trees may be on the order of 10²-10⁵ trees, though there may be diminishing marginal returns to performance metrics (potentially outweighed by computational requirements) beyond the use of a few hundred trees. Once these two models are produced the shape of their Receiver Operator Characteristic Curves (ROC) and respective Areas Under Curve (AUC) can be compared. Optionally, model performance using Vickers and Elkins' Decision Curve Analysis (DCA) and confusion matrices can be assessed. Optionally, both the decision curves of the full variable random forest model and the reduced variable random forest model can be plotted. DCA can be used assess the net benefit of using the models in a clinical setting as compared to the null model, treat no one, or the “treat-all” intervention paradigm.

In some embodiments, clinical parameters including Blood Bethesda, RBC (Red Blood Cells) Bethesda (both measures of volume of blood product received at WRNMMC), Critical Colonization (presence of greater than 10⁶ CFU per gram of tissue or per μl of wound effluent), serum IL2R, and serum MIG outperform other sets of variables. For example, in some embodiments, random forest modeling and ROC/AUC analyses show the full variable model with an AUC of 0.721 or greater, and show the MMPC-selected variable model with an AUC of 0.834 or greater. The sensitivity of the later model may be. 500 or greater, whereas the specificity may be 0.912 or greater. In some embodiments, 50% or more of bacteremia positive cases can be predicted with a model as described herein. In some embodiments, DCA curves show a measurable net benefit of both the full and MMPC-selected variable models as described herein.

### D. Methods for Determining Risk, Detecting Biomarkers, and Treatment

In some embodiments, the methods disclosed herein relate to determining a subject's risk profile for bacteremia, determining if a subject has an increased risk of developing bacteremia, assessing risk factors in a subject, detecting levels of biomarkers, and treating a subject for bacteremia. In accordance with any embodiments of the methods described herein, the subject may be assessed prior to the detection of symptoms of bacteremia, such as prior to detection of the presence of bacteria in the subject's blood system. In accordance with any embodiments of the methods described herein, the test subject may be assessed prior to the onset of any detectable symptoms of bacteremia, such as prior to the subject having a detectable level of bacteria in the subject's blood system detectable by one or more methodologies set forth above. In accordance with any embodiments of the methods described herein, the test subject may have an injury, condition, or wound that puts the subject at risk of developing bacteremia, such as a blast injury, a crush injury, a gunshot wound, or an extremity wound.

### Methods of Detecting Risk Factors

In accordance with some embodiments, there are provided methods of assessing risk factors (e.g., clinical parameters) in a subject, the methods comprising, consisting of, or consisting essentially of measuring, assessing, detecting, assaying, and/or determining one or more clinical parameters, such as one or more selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-Iα) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (pRBCs) administered to the subject, amount of platelets administered to the subject, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, and AIS of skin.

In specific embodiments, there are provided methods of assessing risk factors (e.g., clinical parameters) in a subject, the methods comprising, consisting of, or consisting essentially of measuring, assessing, detecting, assaying, and/or determining one or more clinical parameters, such as one or more selected from a presence of CC in a sample from the subject, level of total RBCs administered to the subject, summation of all blood products administered to the subject, level of IL-2R in a serum sample from the subject, and level of MIG in a serum sample from the subject. In some embodiments, the IL-2R is soluble IL-2R.

In accordance with some embodiments, there are provided methods of detecting levels of biomarkers, the methods comprising, consisting of, or consisting essentially of measuring, detecting, assaying, or determining in one or more samples from the subject levels of one or more biomarkers selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-Iα) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, and level of vascular endothelial growth factor (VEGF) in a sample from the subject.

In specific embodiments, there are provided methods of detecting levels of biomarkers, the methods comprising, consisting of, or consisting essentially of measuring, detecting, assaying, or determining in one or more samples from the subject levels of one or more biomarkers selected from IL-2R, IL-3, IL-8, IL-6 and MIG. In specific embodiments, the one or more biomarkers comprise, consist of, or consist essentially of levels of IL-2R and MIG.

In specific embodiments of any of these methods, one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above are measured, assessed, detected, assayed, and/or determined. In particular embodiments, 2, 3, 4, or 5 clinical parameters are measured, assessed, detected, assayed, and/or determined.

To assay, detect, measure, and/or determine levels of individual clinical parameters, one or more samples is taken or isolated from the subject. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 samples are taken or isolated from the subject. The one or more samples may or may not be processed prior assaying levels of the factors, risk factors, biomarkers, clinical parameters, and/or components. For example, whole blood may be taken from an individual and the blood sample may be processed, e.g., centrifuged, to isolate plasma or serum from the blood. The one or more samples may or may not be stored, e.g., frozen, prior to processing or analysis. In some embodiments, one or more clinical parameters selected from IL-2R, IL-3, IL-6, and/or MIG are detected in a sample from a subject that is not a serum sample, such as a plasma sample or wound effluent.

In some embodiments, levels of individual biomarkers in a sample isolated from a subject are assessed, detected, measured, and/or determined using mass spectrometry in conjunction with ultra-performance liquid chromatography (UPLC), high-performance liquid chromatography (HPLC), gas chromatography (GC), gas chromatography/mass spectroscopy (GC/MS), or UPLC. Other methods of assessing biomarkers include biological methods, such as but not limited to ELISA assays, Western Blot, and multiplexed immunoassays. Other techniques may include using quantitative arrays, PCR, RNA sequencing, DNA sequencing, and Northern Blot analysis. Other techniques include Luminex proteomic data, RNAseq, transcriptomic data, quantitative polymerase chain reaction (qPCR) data, and quantitative bacteriology data.

To determine levels of clinical parameters, particularly biomarkers, it is not necessary that an entire biomarker molecule, e.g., a full length protein or an entire RNA transcript, be present or fully sequenced. In other words, determining levels of, for example, a fragment of protein being analyzed may be sufficient to conclude or assess that an individual component of the risk profile being analyzed is increased or decreased. Similarly, if, for example, arrays or blots are used to determine biomarker levels, the presence, absence, and/or strength of a detectable signal may be sufficient to assess levels of biomarkers.

IL-2Ra antibodies suitable for use in ELISA assays, Western Blots, immunocytochemistry, and immunofluorescence, are available from, for example, Biorbyt (cat# orb161444). IL-2Rb antibodies suitable for use in ELISA assays and Western Blots, are available from, for example, Biorbyt (cat# orb161445). MIG antibodies suitable for use in ELISA assays, immunohistochemistry, and/or Western Blots are available, for example, from Abcam (cat# ab9720). IL-3 antibodies suitable for use in ELISA assays, are available from, for example, ThermoFisher Scientific (cat# AHC0939). IL-3 antibodies suitable for use in Western Blots, immunofluorescence, and immunocytochemistry, are available from, for example, ThermoFisher Scientific (cat# PAS-46918). IL-8 antibodies suitable for use in ELISA assays, immunofluorescence, immunocytochemistry, and Western Blots are available, for example, from ThermoFisher Scientific (cat# M801). IL-6 antibodies suitable for use in ELISA assays, flow cytometry, and/or Western blots, are available from, for example, ThermoFisher Scientific (cat#701028). In some embodiments, the antibodies comprise a detectable label.

As noted above, biomarkers can be detected, assayed, or measured using the Luminex™ immune assay platform available, for example, from ThermoFisher Scientific. For example the Immune Monitoring 65-Plex Human ProcartaPlex™ Panel (cat# EPX650-10065-901) detects the following targets in a single serum or plasma sample: APRIL, BAFF, BLC (CXCL13), CD30, CD40L, ENA-78 (CXCL5), Eotaxin (CCL11), Eotaxin-2 (CCL24), Eotaxin-3 (CCL26), FGF-2, Fractalkine (CX3CL1), G-CSF (CSF-3), GM-CSF, Gro a, HGF, IFN alpha, IFN gamma, IL-10, IL-12p70, IL-13, IL-15, IL-16, IL-17A (CTLA-8), IL-18, IL-1a, IL-1b, IL-2, IL-20, IL-21, IL-22, IL-23, IL-27, IL-2R, IL-3, IL-31, IL-4, IL-5, IL-6, IL-7, IL-8 (CXCL8), IL-9, IP-10 (CXCL10), I-TAC (CXCL11), LIF, MCP-1 (CCL2), MCP-2 (CCL8), MCP-3 (CCL7), M-CSF, MDC, MIF, MIG (CXCL9), MIP-1 alpha (CCL3), MIP-1 beta (CCL4), MIP-3 alpha (CCL20), MMP-1, NGF beta, SCF, SDF-1a, TNF beta, TNF alpha, TNF-R2, TRAIL, TSLP, TWEAK, and VEGF-A.

In some embodiments, clinical parameters are detected, measured, assayed, assessed, and/or determined in a sample isolated from the subject at different time points, such as before, at a first time point after, and/or at a subsequent time point after the subject contracts an injury, condition, or wound that puts the subject at risk of developing bacteremia, such as a blast injury, a crush injury, a gunshot wound, or an extremity wound. For example, some embodiments of the methods described herein may comprise detecting biomarkers at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods also include embodiments in which the subject is assessed before and/or during and/or after treatment for bacteremia. In specific embodiments, the methods are useful for monitoring the efficacy of treatment of bacteremia, and comprise detecting clinical parameters, such as biomarkers in a sample isolated from the subject, at least one, two, three, four, five, six, seven, eight, nine or 10 or more different time points prior to beginning treatment for bacteremia and subsequently detecting clinical parameters, such as at least one, two, three, four, five, six, seven, eight, nine or 10 or more different time points after beginning of treatment for bacteremia, and determining the changes, if any, in the levels detected. The treatment may be any treatment designed to cure, remove or diminish the symptoms and/or cause(s) of bacteremia.

In accordance with some embodiments, there are provided methods of detecting clinical parameters in a subject, the method comprising, consisting of, or consisting essentially of measuring levels of one or more clinical parameters selected from a presence of CC in a sample from the subject, level of total RBCs administered to the subject, summation of all blood products administered to the subject, level of IL-2R in a serum sample from the subject, level of IL-3 in a serum sample from the subject, level of IL-8 in a serum sample from the subject, level of IL-6 in a serum sample from a subject, and level of MIG in a serum sample from the subject. In some embodiments, the methods comprise detecting elevated or reduced levels or values. As used herein, “elevated” refers to a level or value that is increased relative to a reference level or value. As used herein, “reduced” refers to a level that is decreased relative to a reference value or level. In specific embodiments, the reference subject is the test subject at an earlier time, such as prior to or immediately following incurring an injury that puts the subject at risk for bacteremia and at a time when the reference subject did not have detectable symptoms of bacteremia. In specific embodiments of any of these methods, the reference level or value is a value previously detected, measured, assayed, assessed, or determined for the subject. In other embodiments, the reference level or value is detected, measured, assayed, assessed, or determined for a population of one or more reference subjects at a time when the reference subjects did not have detectable symptoms of bacteremia.

### Methods of Determining or Assessing Bacteremia Risk

In accordance with some embodiments, there are provided methods of determining a risk profile for bacteremia, wherein the risk profile comprises, consists of, or consists essentially of one or more components based on one or more clinical parameters selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-Iα) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (pRBCs) administered to the subject, amount of platelets administered to the subject, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, and AIS of skin. Such methods may comprise, consist of or consist essentially of detecting the one or more clinical parameters for the subject, and calculating the subject's risk profile value from the detected clinical parameters.

In specific embodiments, there are provided methods of determining a risk profile for bacteremia, wherein the risk profile comprises, consists of, or consists essentially of one or more components based on one or more clinical parameters selected from a presence of CC in a sample from the subject, level of total RBCs administered to the subject, summation of all blood products administered to the subject, level of IL-2R in a serum sample from the subject, and level of MIG in a serum sample from the subject. Such methods may comprise, consist of or consist essentially of detecting the one or more clinical parameters for the subject, and calculating the subject's risk profile value from the detected clinical parameters. In some embodiments, IL-2R is soluble IL-2R. In some embodiments, one or more clinical parameters selected from IL-2R, IL-3, IL-6, and/or MIG are detected in a sample from a subject that is not a serum sample, such as wound effluent.

In specific embodiments of any of these methods, the risk profile is calculated from one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above. In particular embodiments, the risk profile is calculated from 2, 3, 4, or 5 clinical parameters such as selected from those set forth above. In specific embodiments, a subject is diagnosed as having an increased risk of suffering from bacteremia if the subject's five, four, three, two or even one of the components or factors herein are at abnormal levels. It should be understood that individual levels of risk factor need not be correlated with increased risk in order for the risk profile value to indicate that the subject has an increased risk of developing bacteremia.

In some embodiments, one or more clinical parameters are detected in a sample from the subject that is a biological fluid or tissue isolated from the subject. Biological fluids or tissues include but are not limited to whole blood, peripheral blood, serum, plasma, cerebrospinal fluid, wound effluent, urine, amniotic fluid, peritoneal fluid, lymph fluids, various external secretions of the respiratory, intestinal, and genitourinary tracts, tears, saliva, white blood cells, solid tumors, lymphomas, leukemias, and myelomas. In specific embodiments of any of these methods, one or more clinical parameters are detected in a sample from the subject selected from a serum sample and wound effluent. In specific embodiments of any of these methods, the sample is a plasma sample from the subject.

In specific embodiments of any of these methods, the risk profile value is based on clinical parameters including one or more selected from a presence of CC in a sample from the subject, level of total RBCs administered to the subject, summation of all blood products administered to the subject, level of IL-2R in a serum sample from the subject, and level of MIG in a serum sample from the subject.

In some embodiments, the measurements of the individual components themselves are used in the risk profile, and these levels can be used to provide a “binary” value to each component, e.g., “elevated” or “not elevated.” Each of the binary values can be converted to a number, e.g., “1” or “0,” respectively.

In some embodiments, the “risk profile value” can be a single value, number, factor or score given as an overall collective value to the individual components of the profile. For example, if each component is assigned a value, such as above, the component value may simply be the overall score of each individual or categorical value. For example, if four components of the risk profile for predicting bacteremia are used and three of those components are assigned values of “+2” and one is assigned values of “+1,” the risk profile in this example would be +7, with a normal value being, for example, “0.” In this manner, the risk profile value could be a useful single number or score, the actual value or magnitude of which could be an indication of the actual risk of developing bacteremia, e.g., the “more positive” the value, the greater the risk of developing bacteremia.

In some embodiments, the “risk profile value” can be a series of values, numbers, factors or scores given to the individual components of the overall profile. In another embodiment, the “risk profile value” may be a combination of values, numbers, factors or scores given to individual components of the profile as well as values, numbers, factors or scores collectively given to a group of components, such as a plasma marker portion. In another example, the risk profile value may comprise or consist of individual values, number or scores for specific component as well as values, numbers or scores for a group of components.

In some embodiments, individual values from the risk profile can be used to develop a single score, such as a “combined risk index,” which may utilize weighted scores from the individual component values reduced to a diagnostic number value. The combined risk index may also be generated using non-weighted scores from the individual component values. In such embodiments, when the “combined risk index” exceeds a specific threshold level, such as may be determined by a range of values developed similarly from a population of one or more control (normal) subjects, the individual may be deemed to have a high risk, or higher than normal risk, of developing bacteremia, whereas maintaining a normal range value of the “combined risk index” would indicate a low or minimal risk of developing bacteremia. In these embodiments, the threshold value may be set by the combined risk index from a population of one or more control (normal) subjects.

In some embodiments, the value of the risk profile can be the collection of data from the individual measurements, and need not be converted to a scoring system, such that the “risk profile value” is a collection of the individual measurements of the individual components of the profile.

In some embodiments, the test subject's risk profile is compared to a reference risk profile. In specific embodiments of any of these methods, a reference risk profile value is calculated from clinical parameters previously detected for the test subject. Thus, the present invention also includes methods of monitoring the progression of bacteremia in a subject, with the methods comprising determining the subject's risk profile at more than one time point. For example, some embodiments of the methods of the present invention will comprise determining the test subject's risk profile at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods described herein also include embodiments in which the subject's risk profile is assessed before and/or during and/or after treatment of bacteremia. In other words, the present invention also includes methods of monitoring the efficacy of treatment of bacteremia and/or the subject's response to bacteremia treatment by assessing the subject's risk profile over the course of the treatment and after the treatment. In specific embodiments, the methods of monitoring the efficacy of treatment of bacteremia comprise determining the subject's risk profile at least one, two, three, four, five, six, seven, eight, nine or 10 or more different time points prior to the receipt of treatment for bacteremia and subsequently determining the subject's risk profile at least one, two, three, four, five, six, seven, eight, nine or 10 or more different time points after beginning of treatment for bacteremia, and determining the changes, if any, in the risk profile of the subject. The treatment may be any treatment designed to cure, remove or diminish the symptoms and/or cause(s) of bacteremia.

In other embodiments, a reference risk profile value is calculated from clinical parameters detected for a population of one or more reference subjects when the reference subjects did not have detectable symptoms of bacteremia. In specific embodiments, the reference risk profile value is calculated from clinical parameters detected for a population of reference subjects having an injury, condition, or wound that puts the subject at risk of developing bacteremia, such as a blast injury, a crush injury, a gunshot wound, or an extremity wound.

The levels or values of the clinical parameters compared to reference levels can vary. In some embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000 fold higher than reference levels or values. In some embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000 fold lower than reference levels or values. In the alternative, the levels or values of the factors or components may be normalized to a standard and these normalized levels or values can then be compared to one another to determine if a factor or component is lower, higher or about the same.

In specific embodiments of any of these methods, an increase in the subject's risk profile value as compared to a reference risk profile value indicates that the subject has an increased risk of developing bacteremia.

In other embodiments, the subject's risk profile is compared to the profile that is deemed to be a “normal” risk profile. To establish a “normal” risk profile, an individual or group of individuals may be first assessed to ensure they have no signs, symptoms or diagnostic indicators that they may have bacteremia. Then, the risk profile of the individual or group of individuals can then be determined to establish a “normal risk profile.” In one embodiment, a normal risk profile can be ascertained from the same subject when the subject is deemed healthy, such as when the subject does not have an injury, condition, or wound that puts the subject at risk of developing bacteremia, such as a blast injury, a crush injury, a gunshot wound, or an extremity wound and/or has no signs, symptoms or diagnostic indicators of bacteremia. In some embodiments, however, a risk profile from a “normal subject,” e.g., a “normal risk profile,” is from a subject who has an injury or wound but has no signs, symptoms or diagnostic indicators that they may have bacteremia, such as a subject who has a chest wound, but has no signs, symptoms or diagnostic indicators of bacteremia, or a head wound but no signs, symptoms or diagnostic indicators of bacteremia, or has at least one wound in an extremity (arm, hand, finger(s), leg, foot, toe(s)), but no signs, symptoms or diagnostic indicators of bacteremia.

Thus, in some embodiments, a “normal” risk profile is assessed in the same subject from whom the sample is taken, prior to the onset of any signs, symptoms or diagnostic indicators that they have bacteremia. For example, the normal risk profile may be assessed in a longitudinal manner based on data regarding the subject at an earlier point in time, enabling a comparison between the risk profile (and values thereof) over time.

In another embodiment, a normal risk profile is assessed in a sample from a different subject or patient (from the subject being analyzed) and this different subject does not have or is not suspected of having bacteremia. In still another embodiment, the normal risk profile is assessed in a population of healthy individuals, the constituents of which display no signs, symptoms or diagnostic indicators that they may have bacteremia. Thus, the subject's risk profile can be compared to a normal risk profile generated from a single normal sample or a risk profile generated from more than one normal sample.

In specific embodiments, a subject is diagnosed as having an increased risk of suffering from bacteremia if the subject's five, four, three, two or even one of the components or factors herein are at abnormal levels.

In accordance with some embodiments, there are provided methods of determining if a subject, optionally a subject having an injury that puts the subject at risk of developing bacteremia, has an increased risk of developing bacteremia, optionally prior to the onset of detectable symptoms thereof, comprising: detecting one or more clinical parameters for the subject selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-1α) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (pRBCs) administered to the subject, amount of platelets administered to the subject, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, and AIS of skin; calculating the subject's risk profile value from the detected clinical parameters; and comparing the subject's risk profile value to a reference risk profile value, wherein an increase in the subject's risk profile value as compared to the reference risk profile value indicates that the subject has an increased risk of developing bacteremia. In some embodiments, the subject has an injury that puts the subject at risk of developing bacteremia. In some embodiments, the increased risk of developing bacteremia is determined prior to the onset of detectable symptoms thereof.

In specific embodiments, there are provided methods of determining if a subject, optionally a subject having an injury that puts the subject at risk of developing bacteremia, has an increased risk of developing bacteremia, optionally prior to the onset of detectable symptoms thereof, comprising: detecting one or more clinical parameters for the subject selected from a presence of CC in a sample from the subject, level of total RBCs administered to the subject, summation of all blood products administered to the subject, level of IL-2R in a serum sample from the subject, and level of MIG in a serum sample from the subject; calculating the subject's risk profile value from the detected clinical parameters; and comparing the subject's risk profile value to a reference risk profile value, wherein an increase in the subject's risk profile value as compared to the reference risk profile value indicates that the subject has an increased risk of developing bacteremia. In some embodiments, the subject has an injury that puts the subject at risk of developing bacteremia. In some embodiments, the increased risk of developing bacteremia is determined prior to the onset of detectable symptoms thereof.

In specific embodiments of any of these methods, the method comprises detecting one or more clinical parameters, two or more clinical parameters, three or more clinical parameters, four or more clinical parameters, or five clinical parameters selected from a presence of CC in a sample from the subject, level of total RBCs administered to the subject, summation of all blood products administered to the subject, level of IL-2R in a serum sample from the subject, and level of MIG in a serum sample from the subject.

The present disclosure also provides methods of treating individuals determined to have an increased risk of developing bacteremia for bacteremia, optionally before the onset of detectable symptoms thereof, such as before there are perceivable, noticeable or measurable signs of bacteremia in the individual. Examples of treatment may include initiation or broadening of antibiotic therapy. Benefits of such early treatment may include avoidance of sepsis, avoidance of empyema, avoidance of need for ventilation support, reduced length of stay in hospital or intensive care unit, and/or reduced medical costs.

In accordance with some embodiments, there are provided methods of assessing risk factors in a subject, optionally a subject having an injury that puts the subject at risk of developing bacteremia, comprising assessing one or more risk factors selected from a presence of CC in a sample from the subject, level of total RBCs administered to the subject, summation of all blood products administered to the subject, level of IL-2R in a serum sample from the subject, and level of MIG in a serum sample from the subject. In some embodiments, the risk factors are bacteremia risk factors, and optionally are assessed before the onset of detectable symptoms thereof. In some embodiments, the subject has an injury that puts the subject at risk of developing bacteremia. In some embodiments, the increased risk of developing bacteremia is determined prior to the onset of detectable symptoms thereof.

In accordance with some embodiments, there are provided methods of determining if a subject has an increased risk of developing bacteremia, optionally prior to the onset of detectable symptoms thereof, the method comprising, consisting of, or consisting essentially of: (a) analyzing at least one sample from the subject to determine a value of the subject's risk profile, wherein the risk profile comprises a presence of CC in a sample from the subject, level of total RBCs administered to the subject, summation of all blood products administered to the subject, level of IL-2R in a serum sample from the subject, and level of MIG in a serum sample from the subject, and (b) comparing the value of the subject's risk profile a normal risk profile, to determine if the subject's risk profile is altered compared to a normal risk profile, wherein an increase in the value of the subject's risk profile is indicative that the subject has an increased risk of developing bacteremia compared to individuals with a normal risk profile. In specific embodiments of any of these methods, the normal risk profile comprises a risk profile generated from a population of healthy individuals that do not presently or in the future display symptoms of bacteremia.

In specific embodiments of any of these methods, the risk profile further comprises or consists of the mechanism of injury, level of IL-3 in a serum sample from the subject, level of IL-8 in a serum sample from the subject, and level of IL-6 in a serum sample from the subject.

In some embodiments, such as for univariate analysis, a Wilcoxon rank-sum test can be used to identify which biomarkers from specific patient groups are associated with a specific indication. The assessment of the levels of the individual components of the risk profile can be expressed as absolute or relative values and may or may not be expressed in relation to another component, a standard, an internal standard or another molecule or compound known to be in the sample. If the levels are assessed as relative to a standard or internal standard, the standard or internal standard may be added to the test sample prior to, during or after sample processing.

### Methods of Treating Bacteremia

In accordance with some embodiments, there are provided methods of treating a subject for bacteremia, optionally having an injury that puts the subject at risk for bacteremia, comprising administering a treatment for bacteremia to the subject prior to the onset of detectable symptoms thereof, wherein the subject previously has been determined to have an elevated risk of developing bacteremia as determined by a risk profile value calculated from one or more clinical parameters selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-Iα) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (pRBCs) administered to the subject, amount of platelets administered to the subject, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, and AIS of skin. In some embodiments, the subject has an injury that puts the subject at risk of developing bacteremia. In some embodiments, the increased risk of developing bacteremia is determined prior to the onset of detectable symptoms thereof.

In accordance with some embodiments, there are provided methods of treating a subject for bacteremia, optionally having an injury that puts the subject at risk for bacteremia, comprising administering a treatment for bacteremia to the subject prior to the onset of detectable symptoms thereof, wherein the subject previously has been determined to have an elevated risk of developing bacteremia as determined by a risk profile value calculated from one or more clinical parameters selected from a presence of CC in a sample from the subject, level of total RBCs administered to the subject, summation of all blood products administered to the subject, level of IL-2R in a serum sample from the subject, and level of MIG in a serum sample from the subject. In some embodiments, IL-2R is soluble IL-2R. In some embodiments, the subject has an injury that puts the subject at risk of developing bacteremia. In some embodiments, the increased risk of developing bacteremia is determined prior to the onset of detectable symptoms thereof.

An “elevated risk” refers to a risk profile value of the test subject that is greater than a reference risk profile value (as described above). In some embodiments, an elevated risk is a risk profile value of the test subject that is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000 fold greater than the reference risk profile value.

In accordance with some embodiments, there are provided methods of treating a subject for bacteremia, the method comprising, consisting of, or consisting essentially of: (a) assessing a risk profile comprising individual risk factors selected from: a presence of CC in a sample from the subject, level of total RBCs administered to the subject, summation of all blood products administered to the subject, level of IL-2R in a serum sample from the subject, and level of MIG in a serum sample from the subject, level of IL-3 in a serum sample from the subject, level of IL-8 in a serum sample from the subject, and level of IL-6 in a serum sample from the subject, and (b) administering a treatment for bacteremia to the subject when the risk profile for the subject is greater than the risk profile of a normal subject. In some embodiments, the level of one or more risk factors selected from IL-2R, IL-3, IL-6, and/or MIG is in a sample from a subject that is not a serum sample, such as a plasma sample or wound effluent.

In specific embodiments of any of these methods, the risk profile value is based on clinical parameters including one or more further clinical parameters selected from a presence of CC in a sample from the subject, level of total RBCs administered to the subject, summation of all blood products administered to the subject, level of IL-2R in a serum sample from the subject, and level of MIG in a serum sample from the subject.

In specific embodiments of any of these methods, one or more clinical parameters are detected in a sample from the subject selected from a serum sample and wound effluent. In specific embodiments of any of these methods, the sample is a plasma sample.

In specific embodiments of any of these methods, the reference risk profile value is calculated from clinical parameters previously detected for the subject at a time the subject has the injury.

In specific embodiments of any of these methods, the treatment is administered to the subject prior to the onset of any detectable symptoms of the subject having bacteremia.

The methods of treatment also may include methods of monitoring the effectiveness of a treatment for bacteremia. Once a treatment regimen has been established, with or without the use of the methods of the present disclosure to assist in a prediction of bacteremia or in predicting a risk of developing bacteremia, the methods of monitoring a subject's risk profile over time can be used to assess the effectiveness of treatments for bacteremia. For example, the subject's risk profile can be assessed over time, including before, during and after treatments for bacteremia. The risk profile can be monitored, with, for example, the normalization or decline in the values of the profile over time being indicative that the treatment may be showing efficacy of treatment.

Suitable treatments for bacteremia that may be initiated in response to an indication that the subject is at risk of suffering from bacteremia include but are not limited to administration of antibiotics to the subject.

The present disclosure also provides an antibiotic, for treating bacteremia in a subject having an injury that puts the subject at risk of developing bacteremia, prior to the onset of detectable symptoms thereof, wherein the subject previously has been determined to have an elevated risk of developing bacteremia as determined by any one of the methods described herein.

The present disclosure also provides an antibiotic for use in the preparation of a medicament for treating bacteremia in a subject having an injury that puts the subject at risk of developing bacteremia, prior to the onset of detectable symptoms thereof, wherein the subject previously has been determined to have an elevated risk of developing bacteremia as determined any one of the methods described herein.

The choice of antibiotic or antiviral usually is based on the severity of the subject's illness, host factors (e.g., comorbidity, age), and the presumed causative agent (e.g., species of bacteria). Non-limiting examples of antibiotics include Amoxicillin (Amoxil, Biomox, Trimox), Ampicillin (Marcillin, Omnipen, Polycillin, Principen, Totacillin), Ceftriaxone (Rocephin), Cefotaxime (Claforan), Gentamicin (Garamycin, I-Gent, Jenamicin), Vancomycin (Vancocin, Vancoled, Lyphocin), Nafcillin (Unipen, Nafcil, Nallpen), Meropenem (Merrem), Impenem and cilastatin (Primaxin), Cefepime (Maxipime), and combinations thereof.

In some embodiments of the treatment methods, an effective amount of an antibiotic is administered to the subject. An “effective amount” is an amount sufficient to effect beneficial or desired results such as alleviating at least one or more symptom of bacteremia. An effective amount as used herein would also include an amount sufficient to delay the development bacteremia, alter the course of a bacteremia symptom, or reverse a symptom of bacteremia. Consistent with this definition, as used herein, the term “therapeutically effective amount” is an amount sufficient to inhibit bacteria growth ex vivo, in vitro or in vivo. Thus, an “effective amount” may vary from patient to patient. However, for any given case, an appropriate “effective amount” can be determined by one of ordinary skill in the art using only routine methodologies. An effective amount can be administered in one or more administrations, applications or dosages.

Success of a treatment regime can be determined or assessed by at least one of the following methods: detecting an improvement in one or more symptoms of bacteremia in the subject, determining that the subject has not developed symptoms of bacteremia following treatment, detecting a reduction in the level or value of one or more components of the subject's risk factor profile, and detecting a reduction in the value of the subject's risk factor profile. In some embodiments, success of a treatment regime can be determined or assessed by detecting an increase in the level or value of the subject's risk factor profile (or of one or more components of the subject's risk factor profile) and/or detecting an increase in the value of the subject's risk factor profile relative to the reference risk factor profile (or one or more corresponding component(s) of the reference risk factor profile). Symptoms of bacteremia include but are not limited to fever, rapid heart rate, shaking chills, low blood pressure, gastrointestinal symptoms, such as but not limited to abdominal pain, nausea, vomiting, and diarrhea, rapid breathing, and/or confusion. If severe enough, bacteremia can lead to sepsis, severe sepsis and possible septic shock. In some embodiments, success of treatment of bacteremia can be determined by performing diagnostic tests on the subject. Diagnostic tests for bacteremia include but are not limited to, White Blood Cell (WBC) count, Absolute Neutrophil Count (ANC), Absolute Band Count (ABC), Erythrocyte Sedimentation Rate (ESR), C-Reactive Protein (CRP) level, Procalcitonin level, Urinalysis and Urine culture, Stool studies for children with diarrhea, Plasma Clearance Rate, Lumbar puncture and cerebrospinal fluid (CSF) analysis and Blood culture.

### Kits

In accordance with some embodiments, there are provided kits for performing any of the methods described herein. Thus, the present invention provides kits for determining a risk profile for bacteremia, for determining if a subject has an increased risk of developing bacteremia, for assessing risk factors in a subject, for determining if a subject has an increased risk of developing bacteremia, for detecting levels of biomarkers in a subject, for detecting elevated levels of biomarkers in a subject, and for treating a subject for bacteremia, as described above.

In some embodiments, the kits comprise, consist of, or consist essentially of one or more reagents for detecting one or more biomarkers, such as one or more sets of antibodies immobilized onto a solid substrate that specifically bind to a biomarker. In specific embodiments, the kits comprise at least two, three, four or five sets of antibodies immobilized onto a solid substrate, with each set being useful for detecting a biomarker discussed herein (e.g., IL-2R, MIG).

In specific embodiments, the antibodies that are immobilized onto the substrate may or may not be labeled. For example, the antibodies may be labeled, e.g., bound to a labeled protein, in such a manner that binding of the specific protein may displace the label and the presence of the marker in the sample is marked by the absence of a signal. In addition, the antibodies that are immobilized onto the substrate may be directly or indirectly immobilized onto the surface. Methods for immobilizing proteins, including antibodies, are well-known in the art, and such methods may be used to immobilize a target protein, e.g., IL-2R, or another antibody onto the surface of the substrate to which the antibody directed to the specific factor can then be specifically bound. In this manner, the antibody directed to the specific biomarker is immobilized onto the surface of the substrate for the purposes of the present invention.

IL-2Ra antibodies suitable for use in ELISA assays, Western Blots, immunocytochemistry, and immunofluorescence, are available from, for example, Biorbyt (cat# orb161444). IL-2Rb antibodies suitable for use in ELISA assays and Western Blots, are available from, for example, Biorbyt (cat# orb161445). MIG antibodies suitable for use in ELISA assays, immunohistochemistry, and/or Western Blots are available, for example, from Abcam (cat# ab9720). IL-3 antibodies suitable for use in ELISA assays, are available from, for example, ThermoFisher Scientific (cat# AHC0939). IL-3 antibodies suitable for use in Western Blots, immunofluorescence, and immunocytochemistry, are available from, for example, ThermoFisher Scientific (cat# PAS-46918). IL-8 antibodies suitable for use in ELISA assays, immunofluorescence, immunocytochemistry, and Western Blots are available, for example, from ThermoFisher Scientific (cat# M801). IL-6 antibodies suitable for use in ELISA assays, flow cytometry, and/or Western blots, are available from, for example, ThermoFisher Scientific (cat#701028). In some embodiments, the antibodies comprise a detectable label.

In some embodiments, the kits of the present disclosure comprise, consist of, or consist essentially of containers for collecting samples from the subject and one or more reagents, e.g., one or more antibodies useful for detecting IL-2R or MIG, and/or a purified target biomarker for preparing a calibration curve.

In some embodiments, the kits further comprise additional reagents such as wash buffers, labeling reagents and reagents that are used to detect the presence (or absence) of a label.

In some embodiments, the kits further comprise instructions for use.

### E. Computing Environment

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “engine,” “module,” or “system.” Furthermore, aspects of the present disclosure may take the form of a computer program product embodied in one or more computer readable medium(s) having computer readable program code embodied thereon. Aspects of the present disclosure may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various input/output, control, analysis and other functions described herein, such as by executing instructions of a computer program product.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure may be implemented using various software environments, including but not limited to SAS and R package. SAS (“statistical analysis software”) is a general purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis. R package is free, general purpose package that complies with and runs on a variety of UNIX platforms.

Aspects of the present disclosure are described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In some embodiments, the systems described herein, such as COPS 100 and/or remote device 150, include communications electronics. The communications electronics can be configured to transmit and receive electronic signals from a remote source, such as another electronic device, a cloud server, or an Internet resource. The communications electronics 120 can be configured to communicate using any number or combination of communication standards (e.g., Bluetooth, GSM, CDMA, TDNM, WCDMA, OFDM, GPRS, EV-DO, WiFi, WiMAX, S02.xx, UWB, LTE, satellite, etc). The communications electronics may also include wired communications features, such as USB ports, serial ports, IEEE 1394 ports, optical ports, parallel ports, and/or any other suitable wired communication port.

In some embodiments, the systems described herein, such as the COPS 100 and/or remote device, 150, include a user interface device including a display device and a user input device. The display device may include any of a variety of display devices (e.g., CRT, LCD, LED, OLED) configured to receive image data display the image data. For example, image data can be used to display predictions of bacteremia outcomes. The user input device can include various user interface elements such as keys, buttons, sliders, knobs, touchpads (e.g., resistive or capacitive touchpads), or microphones. In some embodiments, the user interface device includes a touchscreen display device and user input device, such that the user interface device can receive user inputs as touch inputs and determine commands indicated by the user inputs based on detecting location, intensity, duration, or other parameters of the touch inputs.

The construction and arrangement of the systems and methods as shown in the various exemplary embodiments are illustrative only. Although only a few embodiments have been described in detail in this disclosure, many modifications are possible (e.g., variations in sizes, dimensions, structures, and proportions of the various elements, values of parameters, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

Although the figures show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

### Example 1

This example describes an observational study in which 73 patients with injuries were enrolled. Patients required a median of three operations subsequent to enrollment. The incidence of bacteremia was 22% in the patient cohort. Patients required a median of 3 operations subsequent to enrollment. The dataset includes 116 wounds and 399 data collection time points. All modeling results were generated using the first available time point of data, a median of 5 days. Models were also generated using systemic and clinical markers per patient.

Patients with complex wounds cared for at Walter Reed National Military Medical Center (WRNMMC) had data collected prospectively in this observational study. This study was approved by the institutional review board at the primary institution. Tissue, serum and wound effluent samples were collected at all relevant operative interventions from time of consent until wound closure. All wounds were managed with negative pressure dressings allowing molecular assessment of wound dynamics. At each of the time points clinical parameters including both clinical and biomarker data were collected. Clinical parameter data points included gender, age, date location and mechanism of injury, requirement for transfusion and total number of blood products, injury severity score (ISS) and Acute Physiology and Chronic Health Evaluation II (APACHE II) score, wound surface area and depth, associated injuries, type of and success of wound closure, Glasgow Coma Scale (GCS) score, presence and severity of traumatic brain injury, intensive care unit and hospital length of stay, ventilator days, number of wound debridements, development of nosocomial infections and disposition from hospital.

Collection of biomarker data included Luminex proteomic, quantitative PCR (QPCR) transcriptomic, and quantitative bacteriology data. This data was gathered on both serum and wound effluent samples for QPCR and Luminex, whereas quantitative bacteriology assessments were conducted on wound tissue and effluent samples. To extract the most predictive and clinical value for the earliest possible diagnosis and risk prediction of onset of bacteremia in the patient cohort, a subset of the dataset was created with only the first available time point.

Techniques of blood collection and serum and wound inflammatory biomarker analysis have been published elsewhere. See Stojadinovic A., et al., J. Multidiscip Healthc. 3:125-35 (2010), which is incorporated by reference. In brief, blood was collected, fractionated immediately using a centrifuge and plasma supernatant was flash frozen in liquid nitrogen and stored at −70° C. Serum was then analyzed using a BEADLYTE® Human 22-Plex Multi-Cytokine Detection System on the LUMINEX® 100 IS xMAP Bead Array Platform (Millipore Corp). Twenty-two cytokines were quantified in pg/mL according to manufacturer's instructions. Effluent from negative pressure containers were handled similarly.

In this specific study, bacteremia was defined as the presence of bacteria in the blood at any point during the study period that was treated with antibiotics. Clinical end points were determined through chart reviews of enrolled patients.

To perform variable selection on the entire set of serum and effluent variables as well as available clinical variables a “bnlearn” R package(1) was employed. Several algorithms were used to search the input dataset with ranked predictors to find a reduced variable set that best represented the underlying distribution of all variables with respect to the infectious complication outcomes. A feature selection filter algorithm was used to choose the reduced variable set. These algorithms included the inter.iamb, fast.iamb, iamb, gs, mmpc, and si.hiton.pc algorithms. While all algorithms were examined for testing, the Maximum Minimum Parents Children (mmpc) or the inter.iamb algorithm was used to choose the nodes of the corresponding Bayesian network as the reduced variable set. Once each algorithm selected variables for inclusion, a Bayesian network was constructed, and the quality of statistical models was compared using the Bayesian Information Criterion (BIC). The models with the largest BIC were flagged for further modeling and analysis.

Next, a random forest model was constructed using the full set of variables pulled from the raw data as a baseline. To handle process samples with missing data, R packages rfImpute was used. The total, positive class and negative class out-of-bag (00B) error estimates of the model were plotted and then the Accuracy and Kappa scores were calculated. The “randomForest” R package was used for these calculations. This full set of variables was the same full set from which variables were selected. Next, a random forest model was constructed with the Bayesian network-selected variables pulled from the raw data. In addition, the random forest performance with OOB error plots, Accuracy and Kappa scores were assessed. The model with the smallest OOB errors and BIC scores and the highest Accuracy and Kappa scores were chosen. Both random forest models were constructed using 10001 classification and regression trees and square root of p variables randomly sampled as candidates at each split, where p is the number of variables in the model. Once these two models were produced the shape of their Receiver Operator Characteristic Curves (ROC) and respective Areas Under Curve (AUC) were compared. Model performance using Vickers and Elkins' Decision Curve Analysis (DCA) and confusion matrices were also assessed. Both the decision curves of the full variable random forest model and the reduced variable random forest model were also plotted. DCA was used to assess the net benefit of using the models in a clinical setting as compared to the null model, treat no one, or the “treat-all” intervention paradigm.

### Results

The Bayesian network with the best properties above was the mmpc-selected variable set. This model included the following variables: Blood Bethesda, RBC (Red Blood Cells) Bethesda (both measures of volume of blood product received at WRNMMC), Critical Colonization (presence of greater than 10⁶ CFU per gram of tissue or per μl of wound effluent), serum IL2R, and serum MIG. The results of random forest modeling and ROC/AUC analyses show the full variable model with an AUC of 0.721, and show the mmpc-selected variable model with an AUC of 0.834. The sensitivity of the later model was 0.500, whereas the specificity was 0.912. This demonstrates that 50% of the bacteremia positive cases were predicted with this model. DCA curves also show a measurable net benefit of both the full and mmpc-selected variable models.

### Example 2

The process by which a random forest is used to make predictions of bacteremia status is illustrated with reference to FIG. 3. FIG. 3 depicts a single member of the random forest—it is a decision tree, or CART (classification and regression tree), that illustrates the process by which a random forest is used to make predictions of bacteremia status using the clinical parameters: Blood_Bethesda, RBC_Bethesda, Ser2x_IL2R, Ser2x_MIG, and CC. For details on how CARTs and random forests are constructed, refer to “Classification and Regression Trees” (Breiman et al., 1984) and “Random Forests” (Breiman, 2001). The original patient dataset is divided into training and test observations; the training observations are used to construct the random forest.

Test observations are used to make outcome predictions (presence/absence of bacteremia). To illustrate the prediction process with a single tree, we begin with a hypothetical patient X with the following clinical parameter values: Blood_Bethesda of 22, Ser2x_IL2R of 55, RBC_Bethesda of 65, Ser2x_MIG of 30, and CC of Yes. In the decision tree shown in FIG. 3, the first split point consists of the rule: “Is Blood_Bethesda<44.75?” For patient X, the answer is YES, so the next decision encountered decision rule is “Is Ser2x_IL2R<52?”. The process continues until patient X reaches a decision rule that is followed by two terminal nodes: “bacteremia” and “well.” The assignment of outcome to a terminal node is determined by the majority vote of training observations that fall into that node. If a terminal node contains more training subjects with bacteremia, then a test subject that falls into that node and will be predicted to have bacteremia.

Predictions are combined across all the trees in the random forest and the probability of having bacteremia is determined. For example, if there are 10000 trees in the random forest and 5000 trees predict bacteremia, then the assigned probability of having bacteremia is 0.5.

All patents and publications mentioned in this specification are indicative of the level of those skilled in the art to which the present disclosure pertains. All patents and publications cited herein are incorporated by reference to the same extent as if each individual publication was specifically and individually indicated as having been incorporated by reference in its entirety.

### LIST OF REFERENCES THAT ARE INCORPORATED BY REFERENCE

Brown R B, Hosmer D, Chen H C, Teres D, Sands M, Bradley S, Opitz E, Szwedzinski D, Opalenik D. A comparison of infections in different ICUs within the same hospital. Crit Care Med. 1985 June; 13(6): 472-6.
Poole G V, Muakkassa F F, Griswold J A. The role of infection in outcome of Multiple Organ Failure. Am Surg. 1993 November 59(11): 727-32.
Jarvis W R, Edwards J S, Culver D H, Hughes J M, Horan T, Emori T G, Banerjee S,
Tolson J, Henderson T, Gaynes R P, et al. Nosocomial infection rates in adult and pediatric intensive care units in the United States. National Nosocomial Infections Surveillance System. Am J Med. 1991 Sep. 16; 91 (3B): 185S-191S.
Improving Diagnosis in Health Care. Committee on diagnostic error in health care, Board on health care services, Institute of medicine, The national academies of sciences, Engineering and Medicine. IN: Balogh E P, Miller B T, Ball J R, editors. Washington, D C: National Academies Press (US); 2015.
Chromy B A, Eldridge A, Forsberg J A, Brown T S, Kirkup B C, Jaing C, Be N A, Elster E, Luciw P A. Wound outcome in combat injuries is associated with a unique set of protein biomarkers. J Transl Med. 2013 November; 11: 281.
Hawksworth J S, Stojadinovic A, Gage F A, TAdaki D K, Perdue P W, Forsberg J, Davis T A, Dunne J R, Denobile J W, Brown T S, Elster E A. Inflammatory biomarkers in combat wound healing. Ann Surg. 2009 December; 250(6): 1002-7.
Stojadinovic A, Eberhardt J, Brown T S, Hawksworth J S, Gage F, Tadaki D K, Forsberg J A, Davis T A, Potter B K, Dunne J R, Elster E A. Development of a Bayesian model to estimate health care outcomes in the severely wounded. J Multidiscip Healthc. 2010; 3: 125-35.
Ledley R, Lugsted. Reasoning foundations of medical diagnosis; system logic, probability, and value theory aid our understanding of how physicians reason. Science. 1959; 130(3366): 9-21.
Shortliffe E, Davis R, Axkline S, Buchanan B, Green C, Cohen S. Computer-based consultations in clinical therapeutics: explanations and rule acquisition capabilities of the MYCIN system. Comput Biomed Res. 1975; 8(4): 303-20.
Gorry G, Barnett G. Sequential diagnosis by computer. JAMA. 1968; 205(12): 849-54.
Sheppard L, Kouchoukos N, Kurtss M, Kirklin J. Automated treatment of critically ill patients following operation. Ann Surg. 1968; 168(4):596-604.
Ingraham A, Cohen M, Bilimoria K, Dimick J, Richards K, Raval M, Fleisher L A, Hall B L, Ko C Y. Association of surgical care improvement project infection-related process measure compliance with risk-adjusted outcomes: Implications for quality measurement. J Am Coll Surg. 2010 December (6): 705-14.
Eslami S, Abu-Hanna A, de Keiser N. Evaluation of outpatient computerized physician medication order entry systems: A systematic review. J Am Med Inform Assoc. 2007; 14(4): 400-6.
Friedman C, Elstein A, Wolf F, Murphy G, Franz T, Heckerling P, Fine P L, Miller T M, Abraham V. Enhancement of clinicians' diagnostic reasoning by computer-based consultation: A multisite study of 2 systems. JAMA. 1999 November; 282(19): 1851-6.
Samore M H, Bateman K, Alder S C, Hannah E, Donnelly S, Stoddard G J, Haddadin B, Rubin M A, Williamson J, Stults B, et al. Clinical decision support and appropriateness of antimicrobial prescribing: A randomized trial. JAMA. 2005 November; 294 (18): 2305-14.
Graber M, Mathew A. Performance of a web-based clinical diagnosis support system for internists. J Gen Intern Med. 2088; 23(Suppl):37-40.
Sng B, Tan H, Sia A. Closed-loop double-vasopressor automated system vs. manual bolus vasopressor to treat hypotension during spinal anaesthesia for caesarean section: A randomized controlled trial. Anaesthesia. 2014 69(1): 37-45.
Uemura K, Kawada T, Zheng C, Sugimachi M. Less invasive and inotrope-reduction approach to automated closed-loop control of hemodynamics in decompensated heart failure. IEEE Trans Biomed Eng. 2015.
Valenzuela-Sanchez F, Valenzuela-Mendez B, Rodriquez-Gutierrez J F, Estella-Garcia A, Gonzalez-Garcia M A. New role of biomarkers: Mid-regional pro-adrenomedullin, the biomarker of organ failure. Ann Transl Med. 2016 September; 4(17): 329.
He Y, Du W X, Jiang H Y, Ai Q, Fen J, Lui Z, Yu J L. Multiplex cytoking profiling identifies Interleukin-27 as a novel biomarker for neonatal early onset sepsis. Shock Epub 2016 Sep. 19.
Huang L, Li J, Han Y, Zhao S, Zheng Y, Sui F, Xin X, Ma W, Jiang Y, Yao Y, Li W. Serum calprotectin expression as a diagnostic marker for sepsis in postoperative intensive care unit patients. J Interferon Cytokine Res. 2016 October; 36(10): 607-16.
Forsberg J A, Potter B K, Wagner M B, Vickers A, Dente C J, Kirk A D, Elster E A. Lessons of war: Turning data into decisions. EBioMedicine 2015 July; 2(9): 1235-42.
Tojo M, Yamashita N, Golmann D A, Pier G B. Isolation and characterization of a capsular polysaccharide adhesion from Staphyloccocus epidermidis. J Infect Dis. 1988; 157: 713-22.

1.-37. (canceled)

38. A method of generating a model for predicting a bacteremia outcome in a subject comprising:
generating a training database storing first values of a plurality of clinical parameters and bacteremia outcomes associated with a plurality of first subjects;
executing a plurality of variable selection algorithms to select a subset of model parameters from the plurality of clinical parameters for each variable selection algorithm;
executing each one of a plurality of classification algorithms for one of the plurality of subsets of model parameters to generate predictions of bacteremia outcome;
calculating a performance metric associated with each of the plurality of classification algorithms in accordance with the predictions of bacteremia outcome;
selecting a candidate classification algorithm in accordance with the performance metric; and
outputting a model for predicting a bacteremia outcome, the model comprising the candidate classification algorithm with associated subset of model parameters.

39. The method of claim 38, further comprising pre-processing data that is stored in the training database including:
determining that a first value of at least one of the plurality of clinical parameters is missing;
estimating a reference value for the at least one of the plurality of clinical parameters that is missing; and
storing the reference value as the first value of the at least one of the plurality of clinical parameters in the training database.

40. The method of claim 38, wherein the plurality of variable selection algorithms comprise at least one of machine learning algorithm, supervised machine learning algorithm, Grow-Shrink algorithm, Incremental Association Markov Blanket algorithm, or Semi-Interleaved Hiton-PC algorithm.

41. The method of claim 38, wherein the classification algorithm comprises at least one of linear discriminant analysis, classification and regression tree, decision tree learning, random forest model, nearest neighbor, support vector machine, logistic regression, generated linear model, Bayesian model, or neural network.

42. The method of claim 38, wherein selecting a candidate classification algorithm in accordance with the performance metric further comprises:
executing decision curve analysis (DCA) with each classification algorithm, the DCA indicating a net benefit of providing a treatment based on bacteremia outcomes generated by the classification algorithm; and
selecting the classification algorithm having a largest net benefit of providing the treatment as the candidate classification algorithm.

43. The method of claim 38, further comprising:
cross-validating performances of the plurality of classification algorithms.

44. The method of claim 38, wherein the performance metric associated with each of the plurality of classification algorithms includes at least one of a total out-of-bag (OOB) error estimate, a positive class OOB error estimate, a negative OOB error estimate, an accuracy score, or a Kappa score.

45. The method of claim 38, wherein the plurality of clinical parameters comprise one or more biomarker clinical parameters, one or more administration of blood products clinical parameters, one or more injury severity score clinical parameters, or a combination thereof.

46. The method of claim 45, wherein
the biomarker clinical parameter comprises one or more of a level of epidermal growth factor (EGF) in a sample from the subject, a level of eotaxin-1 (CCL11) in a sample from the subject, a level of basic fibroblast growth factor (bFGF) in a sample from the subject, a level of granulocyte colony-stimulating factor (G-CSF) in a sample from the subject, a level of granulocyte-macrophage colony-stimulating factor (GM-CSF) in a sample from the subject, a level of hepatocyte growth factor (HGF) in a sample from the subject, a level of interferon alpha (IFN-α) in a sample from the subject, a level of interferon gamma (IFN-γ) in a sample from the subject, a level of interleukin 10 (IL-10) in a sample from the subject, a level of interleukin 12 (IL-12) in a sample from the subject, a level of interleukin 13 (IL-13) in a sample from the subject, a level of interleukin 15 (IL-15) in a sample from the subject, a level of interleukin 17 (IL-17) in a sample from the subject, a level of interleukin 1 alpha (IL-Iα) in a sample from the subject, a level of interleukin 1 beta (IL-Iβ) in a sample from the subject, a level of interleukin 1 receptor antagonist (IL-IRA) in a sample from the subject, a level of interleukin 2 (IL-2) in a sample from the subject a level of interleukin 2 receptor (IL-2R) in a sample from the subject, a level of interleukin 3 (IL-3) in a sample from the subject, a level of interleukin 4 (IL-4) in a sample from the subject, a level of interleukin 5 (IL-5) in a sample from the subject, a level of interleukin 6 (IL-6) in a sample from the subject, a level of interleukin 7 (IL-7) in a sample from the subject, a level of interleukin 8 (IL-8) in a sample from the subject, a level of interferon gamma induced protein 10 (IP-10) in a sample from the subject, a level of monocyte chemoattractant protein 1 (MCP-1) in a sample from the subject, a level of monokine induced by gamma interferon (MIG) in a sample from the subject, a level of macrophage inflammatory protein 1 alpha (MIP-Iα) in a sample from the subject, a level of macrophage inflammatory protein 1 beta (MIP-Iβ) in a sample from the subject, a level of chemokine (C-C motif) ligand 5 (CCL5) in a sample from the subject, a level of tumor necrosis factor alpha (TNFα) in a sample from the subject, or a level of vascular endothelial growth factor (VEGF) in a sample from the subject,
the administration blood products clinical parameter comprises one or more of an amount of whole blood cells administered to the subject, amount of red blood cells (RBCs) administered to the subject, amount of packed red blood cells (pRBCs) administered to the subject, amount of platelets administered to the subject, summation of all blood products administered to the subject, or a level of total packed RBCs, and
the injury severity score clinical parameter comprises one or more of Injury Severity Score (ISS), Abbreviated injury scale (AIS) of abdomen, AIS of chest (thorax), AIS of extremity, AIS of face, AIS of head, or AIS of skin.

47. A method for predicting a bacteremia outcome for a subject comprising:
receiving, from a second subject, a second value of at least one clinical parameter of a plurality of clinical parameters;
executing a pre-trained model for predicting a bacteremia outcome of the second subject using the second value of at least one clinical parameter, wherein the model is pre-trained by performing operations comprising:
generating a training database storing first values of the plurality of clinical parameters and bacteremia outcomes associated with a plurality of first subjects;
executing a plurality of variable selection algorithms to select a subset of model parameters from the plurality of clinical parameters for each variable selection algorithm;
executing each one of a plurality of classification algorithms for one of the plurality of subsets of model parameters to generate predictions of bacteremia outcome;
calculating a performance metric associated with each of the plurality of classification algorithms in accordance with the predictions of bacteremia outcome;
selecting a candidate classification algorithm in accordance with the performance metric; and
outputting a model for predicting the bacteremia outcome, the model comprising the candidate classification algorithm with associated subset of model parameters; and
outputting the predicted bacteremia outcome of the second subject.

48. The method of claim 47, wherein the operations to pre-train the model further comprise pre-processing data that is stored in the training database including:
determining that a first value of at least one of the plurality of clinical parameters is missing;
estimating a reference value for the at least one of the plurality of clinical parameters that is missing; and
storing the reference value as the first value of the at least one of the plurality of clinical parameters in the training database.

49. The method of claim 47, wherein the plurality of variable selection algorithms comprise at least one of machine learning algorithm, supervised machine learning algorithm, Grow-Shrink algorithm, Incremental Association Markov Blanket algorithm, or Semi-Interleaved Hiton-PC algorithm, or backwards limitation.

50. The method of claim 47, wherein the classification algorithm comprises at least one of linear discriminant analysis, classification and regression tree, decision tree learning, random forest model, nearest neighbor, support vector machine, logistic regression, generated linear model, Bayesian model, or neural network.

51. The method of claim 47, wherein selecting a candidate classification algorithm in accordance with the performance metric further comprises:
executing decision curve analysis (DCA) with each classification algorithm, the DCA indicating a net benefit of providing a treatment based on bacteremia outcomes generated by the classification algorithm; and
selecting the classification algorithm having a largest net benefit of providing the treatment.

52. The method of claim 47, further comprising:
cross-validating performances of the plurality of classification algorithms.

53. The method of claim 47, wherein the performance metric associated with each of the plurality of classification algorithms includes at least one of a total out-of-bag (OOB) error estimate, a positive class OOB error estimate, a negative OOB error estimate, an accuracy score, or a Kappa score.

54. The method of claim 47, wherein the plurality of clinical parameters comprise one or more biomarker clinical parameters, one or more administration of blood products clinical parameters, one or more injury severity score clinical parameters, or a combination thereof.

55. The method of claim 54, wherein
the biomarker clinical parameter comprises one or more of a level of epidermal growth factor (EGF) in a sample from the subject, a level of eotaxin-1 (CCL11) in a sample from the subject, a level of basic fibroblast growth factor (bFGF) in a sample from the subject, a level of granulocyte colony-stimulating factor (G-CSF) in a sample from the subject, a level of granulocyte-macrophage colony-stimulating factor (GM-CSF) in a sample from the subject, a level of hepatocyte growth factor (HGF) in a sample from the subject, a level of interferon alpha (IFN-α) in a sample from the subject, a level of interferon gamma (IFN-γ) in a sample from the subject, a level of interleukin 10 (IL-10) in a sample from the subject, a level of interleukin 12 (IL-12) in a sample from the subject, a level of interleukin 13 (IL-13) in a sample from the subject, a level of interleukin 15 (IL-15) in a sample from the subject, a level of interleukin 17 (IL-17) in a sample from the subject, a level of interleukin 1 alpha (IL-Iα) in a sample from the subject, a level of interleukin 1 beta (IL-Iβ) in a sample from the subject, a level of interleukin 1 receptor antagonist (IL-IRA) in a sample from the subject, a level of interleukin 2 (IL-2) in a sample from the subject a level of interleukin 2 receptor (IL-2R) in a sample from the subject, a level of interleukin 3 (IL-3) in a sample from the subject, a level of interleukin 4 (IL-4) in a sample from the subject, a level of interleukin 5 (IL-5) in a sample from the subject, a level of interleukin 6 (IL-6) in a sample from the subject, a level of interleukin 7 (IL-7) in a sample from the subject, a level of interleukin 8 (IL-8) in a sample from the subject, a level of interferon gamma induced protein 10 (IP-10) in a sample from the subject, a level of monocyte chemoattractant protein 1 (MCP-1) in a sample from the subject, a level of monokine induced by gamma interferon (MIG) in a sample from the subject, a level of macrophage inflammatory protein 1 alpha (MIP-Iα) in a sample from the subject, a level of macrophage inflammatory protein 1 beta (MIP-Iβ) in a sample from the subject, a level of chemokine (C-C motif) ligand 5 (CCL5) in a sample from the subject, a level of tumor necrosis factor alpha (TNFα) in a sample from the subject, or a level of vascular endothelial growth factor (VEGF) in a sample from the subject,
the administration blood products clinical parameter comprises one or more of an amount of whole blood cells administered to the subject, amount of red blood cells (RBCs) administered to the subject, amount of packed red blood cells (pRBCs) administered to the subject, amount of platelets administered to the subject, summation of all blood products administered to the subject, or a level of total packed RBCs, and
the injury severity score clinical parameter comprises one or more of Injury Severity Score (ISS), Abbreviated injury scale (AIS) of abdomen, AIS of chest (thorax), AIS of extremity, AIS of face, AIS of head, or AIS of skin.

56. A system for generating a model for predicting a bacteremia outcome in a subject comprising:
one or more processors;
a memory;
a communication platform;
a training database configured to store first values of a plurality of clinical parameters and bacteremia outcomes associated with a plurality of first subjects;
a machine learning engine configured to:
execute a plurality of variable selection algorithms to select a subset of model parameters from the plurality of clinical parameters for each variable selection algorithm;
execute each one of a plurality of classification algorithms for one of the plurality of subsets of model parameters to generate predictions of bacteremia outcome;
calculate a performance metric associated with each of the plurality of classification algorithms in accordance with the predictions of bacteremia outcome;
select a candidate classification algorithm in accordance with the performance metric; and
output a model for predicting a bacteremia outcome, the model comprising the candidate classification algorithm with associated subset of model parameters.

57. A system for predicting a bacteremia outcome in a subject comprising:
one or more processors;
a memory;
a communication platform;
a training database configured to store first values of a plurality of clinical parameters and bacteremia outcomes associated with a plurality of first subjects;
a machine learning engine configured to pre-train a model for a bacteremia outcome of a subject, wherein the model is pre-trained by performing operations comprising:
generating a training database storing first values of the plurality of clinical parameters and bacteremia outcomes associated with a plurality of first subjects;
executing a plurality of variable selection algorithms to select a subset of model parameters from the plurality of clinical parameters for each variable selection algorithm;
executing each one of a plurality of classification algorithms for one of the plurality of subsets of model parameters to generate predictions of bacteremia outcome;
calculating a performance metric associated with each of the plurality of classification algorithms in accordance with the predictions of bacteremia outcome;
selecting a candidate classification algorithm in accordance with the performance metric; and
outputting a model for predicting the bacteremia outcome, the model comprising the candidate classification algorithm with associated subset of model parameters; and
a prediction engine configured to
receive, from a second subject, a second value of at least one clinical parameter of a plurality of clinical parameters; and
execute the pre-trained model for predicting a bacteremia outcome of the second subject using the second value of at least one clinical parameter; and
a display device configured to output the predicted bacteremia outcome of the second subject.

58. A non-transitory computer-readable medium having information recorded thereon for generating a model for predicting a bacteremia outcome in a subject, wherein the information, when read by a computer, causes the computer to perform operations of:
generating a training database storing first values of a plurality of clinical parameters and bacteremia outcomes associated with a plurality of first subjects;
executing a plurality of variable selection algorithms to select a subset of model parameters from the plurality of clinical parameters for each variable selection algorithm;
executing each one of a plurality of classification algorithms for one of the plurality of subsets of model parameters to generate predictions of bacteremia outcome;
calculating a performance metric associated with each of the plurality of classification algorithms in accordance with the predictions of bacteremia outcome;
selecting a candidate classification algorithm in accordance with the performance metric; and
outputting a model for predicting a bacteremia outcome, the model comprising the candidate classification algorithm with associated subset of model parameters.
